(19) United States
(12) Patent Application Publication
(10) Pub. No.: US 2006/0120971 A1
(43) Pub. Date: Jun. 8, 2006

(54) Agents and methods for administration to the central nervous system

(75) Inventors: David Crockford, Newburyport, MA (US); Louis Herlands, Cambridge, MA (US)

Correspondence Address: MYERS BIGEL SIBLEY & SAJOVEC, PO BOX 37428, RALEIGH, NC, 27627, US

(73) Assignee: SIGNUM PHARMACEUTICALS, INC.

(21) Appl. No.: 11/244,489

(22) Filed: Oct. 6, 2005

Related U.S. Application Data
(60) Provisional application No. 60/617,098, filed on Oct. 8, 2004.

(51) Int. Cl. A61K 9/14; A61K 31/19; A61K 48/00; A61K 31/365

(52) U.S. Cl. 424/46; 514/44; 514/557; 514/473

(57) ABSTRACT

The present invention provides pharmaceutical compositions and methods for intranasal administration to a subject to increase long-chain acyl CoA levels in the CNS (e.g., the hypothalamus), to reduce food intake and/or reduce appetite, to improve hepatic autoregulation, and/or to treat a metabolic disorder such as diabetes mellitus, metabolic syndrome, hyperglycemia, insulin resistance, glucose intolerance and/or obesity.

### RELATED APPLICATION INFORMATION

This application claims the benefit of U.S. Provisional Application Ser. No. 60/617,098, filed Oct. 8, 2004, the disclosure of which is incorporated herein by reference in its entirety.

### FIELD OF INVENTION

The present invention is directed to compositions and methods for intranasal delivery to the central nervous system; in particular, the invention is directed to compositions and methods for intranasal delivery to increase levels of long-chain acyl CoAs in the central nervous system.

### BACKGROUND OF THE INVENTION

Diabetes mellitus (also known simply as diabetes) and obesity are considered major health problems particularly in countries of the Western Hemisphere. Diabetes is the only noninfectious disease recognized as epidemic by the World Health Organization (WHO). It can be divided into two major categories, type-1 or insulin-dependent diabetes mellitus or the more common type-2 or noninsulin-dependent diabetes mellitus. The type-2 form of the disease accounts for more than 90% of all cases and is characterized by insulin resistance (insulin utilization defect) and inadequate β-cell activity. Increased fatty acid oxidation in type-2 diabetic patients contributes to their hyperglycemia.

Obesity is the result of an imbalance between energy intake and energy expenditure. It is a major risk factor for diabetes, heart disease, high blood pressure, stroke, sleep apnea, gallstones, some cancers and some forms of arthritis. In the United States about 50 million Americans are obese, according to the National Institute of Diabetes, Digestive and Kidney Diseases (NIDDK), and each year about 300,000 die of obesity-related causes. According to the United States Centers for Disease Control (CDC), the economic cost of obesity was about $117 billion in 2000. The CDC reports that 61% of adults are overweight or obese and 13% of children or adolescents are seriously overweight. This epidemic exacts a steep toll both in terms of lives and costs.

Mammals have the ability to efficiently match caloric intake to caloric expenditure. To accomplish this task, the central nervous system (CNS) monitors the status of peripheral energy stores and ongoing fuel availability. Recent observations support the hypothesis that ongoing food availability can be monitored directly at the CNS level by mechanisms that go well beyond the sensing of glucose. Research on the neuronal control of energy balance began with the observation that lesions in the hypothalamus produce profound increases or decreases in food intake and body weight. The hypothalamus is a gland that regulates eating patterns, body temperature and metabolism. How the hypothalamus receives information as to the amount of fat that a mammal has in store was not well understood. Two theories developed: The lipostatic theory proposed that there was a product of fat metabolism that circulated in the blood and acted as a signal to the hypothalamus, enabling it to monitor the storage and metabolism of fat; whereas the glucostatic theory postulates that hunger and the initiation of eating is the result of the hypothalamus sensing a decline in blood glucose. In fact, the central nervous system does both. More recent work has shown that ingestive behavior is influenced by a distributed network, which includes caudal brainstem, limbic and cortical structures. For example, the CNS monitors the collective status of adipocytes that are dispersed through the body by chemical signals. One such molecule, a protein hormone called leptin, interacts with receptors in the brain directly to signal how much fat is stored in the body. Changes in signal level or activity alter food intake and energy expenditure.

For CNS control of metabolic diseases such as diabetes and obesity, safe, practical and effective methods for preventative or therapeutic intervention are needed. Peripheral routes of administration (e.g., intravascular or oral) may not result in sufficient delivery of the therapeutic agent to the CNS. Further, peripheral routes of administration often result in substantial hepatic metabolism of the therapeutic agent with concomitant loss of activity. Finally, another drawback of peripheral modes of administration is that the therapeutic agent generally exhibits a more widespread distribution throughout the body, which increases the possibility of undesirable side effects due to exposure of peripheral tissues to the therapeutic agent.

Obici et al., (2003) Nature Med. 9:756, circumnavigated the issues associated with peripheral administration of carnitine palmitoyltransferase-1 (CPT1) inhibitors by the central administration of a vector (CPT1L riboplasmid) and two liver/hypothalamic specific CPT1 inhibitors (the reversible CPT1L inhibitor, (R)-N-(tetradecylcarbamoyl)-aminocarnitine) [ST1326] and the CPT1 inhibitor, 2-tetradecylglydate [TDGA]) to decrease levels of CPT1 activity and increase the amount of long-chain fatty acids esterified to Coenzyme A, also known as long-chain acyl-CoA (LC-CoA) in the brain of healthy rats. Local delivery was accomplished by directly administering these genetic and pharmaceutical agents into the third cerebral ventricle of continuously infused, conscious Sprague-Dawley rats by ICV catheters implanted by stereotactic surgical procedures. This local mode of delivering CPT1 inhibitors led to diminished food intake and reduced endogenous glucose production by effectively decreasing CPT1 activity and substantially increasing the hypothalamic concentrations of LC-COA. This mode of administration, however, is disfavored as it is extremely invasive.

Thus, there remains a need in the art for improved compositions and methods for delivering therapeutic agents to the CNS, for example, the brain or the hypothalamus. There is further a need in the art for improved compositions and methods for central treatment of metabolic diseases such as diabetes mellitus, metabolic syndrome and obesity.

### SUMMARY OF THE INVENTION

The present invention provides a method for administering compounds to the central nervous (CNS) system, for example, the brain or the hypothalamus (e.g., the mediobasal hypothalamus including the arcuate nucleus [ARC]), by intranasal delivery to elevate long-chain acyl-CoA (LC-CoA) levels therein, thereby avoiding the need for invasive modes of administration directly to the CNS.

Accordingly, as one aspect, the invention provides a pharmaceutical composition formulated for intranasal administration comprising a compound that elevates LC-CoA levels in the CNS, for example, the brain or the hypothalamus (e.g., the ARC) in a pharmaceutically acceptable carrier. In particular embodiments, the compound reduces or decreases the activity of a LC-CoA-decreasing molecule. In other embodiments, the compound enhances or increases the activity of a LC-CoA-increasing molecule. The composition can optionally be formulated for delivery to the olfactory and/or sinus region of the nose.

As a further aspect, the invention provides a method of elevating LC-CoA levels in the CNS (for example, the brain or the hypothalamus) of a mammalian subject comprising intranasally administering to the mammalian subject an effective amount of a compound or pharmaceutical composition as described herein.

As yet another aspect, the invention provides a method of treating diabetes mellitus in a mammalian subject comprising intranasally administering to the mammalian subject an effective amount of a compound or pharmaceutical composition as described herein.

As still a further aspect, the invention provides a method of treating metabolic syndrome in a mammalian subject comprising intranasally administering to the mammalian subject an effective amount of a compound or pharmaceutical composition as described herein.

As another aspect, the invention provides a method of improving hepatic autoregulation in a mammalian subject comprising intranasally administering to the mammalian subject an effective amount of a compound or pharmaceutical composition as described herein.

As another aspect, the invention provides a method of reducing glucose production in a mammalian subject comprising intranasally administering to the mammalian subject an effective amount of a compound or pharmaceutical composition as described herein.

As a further aspect, the invention provides a method of reducing food intake in a mammalian subject comprising intranasally administering to the mammalian subject an effective amount of a compound or a pharmaceutical composition as described herein.

As still another aspect, the invention provides a method of treating obesity in a mammalian subject comprising intranasally administering to the mammalian subject an effective amount of a compound or pharmaceutical composition as described herein.

In particular embodiments, the compound or pharmaceutical composition is delivered to the olfactory region and/or sinus region.

According to the methods of the invention, the subject can be a human subject or an animal subject including an animal model of diabetes mellitus, metabolic syndrome and/or obesity. Further, in practicing the methods of the invention, the subject can have diabetes mellitus, metabolic syndrome and/or be 20% or more over normal body weight.

The invention further provides methods of identifying compounds for use in the methods of the invention.

Also provided is the use of a compound or pharmaceutical composition of the invention for increasing LC-CoA levels in the CNS (e.g., hypothalamus), treating diabetes, treating metabolic syndrome, reducing glucose production, improving hepatic autoregulation, treating hyperglycemia, treating insulin resistance, treating glucose intolerance, reducing food intake, reducing appetite and/or treating obesity.

These and other aspects of the invention are set forth in more detail in the description of the invention below.

### DETAILED DESCRIPTION OF THE INVENTION

The present invention is based, in part, on the recognition that compounds can be administered intranasally to increase long-chain acyl-CoA (LC-CoA) levels in the CNS, for example, the brain or the hypothalamus (e.g., the ARC), to reduce glucose production and/or food intake, to improve hepatic autoregulation and/or to treat metabolic disorders such as diabetes mellitus, hyperglycemia, insulin resistance, glucose intolerance, metabolic syndrome and/or obesity.

Obici et al., (2003) Nature Med. 9:756, delivered a vector providing a ribozyme directed against CPT1L (CPT1L riboplasmid) and liver/hypothalamic specific CPT1 inhibitors (the reversible CPT1L inhibitor, (R)-N-(tetradecylcarbamoyl)-aminocarnitine [ST1 326] and the CPT1 inhibitor, 2-tetradecylglydate [TDGA]) by central administration to decrease levels of CPT1 activity and increase the levels of LC-CoAs in the hypothalamus of healthy rats. Local delivery was accomplished by direct administration into the third cerebral ventricle of continuously infused, conscious Sprague-Dawley rats by ICV catheters implanted by stereotactic surgical procedures. Central administration of the CPT1 inhibitors resulted in diminished food intake and reduced endogenous glucose production.

The compositions and methods of the present invention provide for the delivery of compounds to the CNS (for example, the brain or the hypothalamus (e.g., the ARC)) by the nasal route, while minimizing systemic exposure. In this regard and without being bound to any particular theory, it is believed that targeting the CNS by nasal administration is based on capture and internalization of substances by the olfactory receptor neurons, which substances are then transported inside the neuron to the olfactory bulb of the brain. Olfactory receptor neurons from the lateral olfactory tract within the olfactory bulb project to various regions such as the hippocampus, amygdala, thalamus, hypothalamus and other regions of the brain that are not directly involved in olfaction. These substances may also pass through junctions in the olfactory epithelium at the olfactory bulb and enter the subarachnoid space, which surrounds the brain, and the cerebral spinal fluid (CSF), which bathes the brain. Either pathway allows for targeted delivery without interference by the blood brain barrier, as neurons and the CSF, not the circulatory system, are involved in these transport mechanisms. Accordingly, intranasal delivery pathways permit compartmentalized delivery of compositions with substantially reduced systemic exposure and the resulting side effects.

As further advantages, nasal delivery offers a noninvasive means of administration that is safe and convenient for self-medication, and which reduces the first-pass hepatic effect (i.e., metabolic degradation by the liver), which can result in greater bioavailability and lower dosages of the therapeutic agent. Intranasal administration can also provide for rapid-onset of action due to rapid absorption by the nasal mucosa. These characteristics of nasal delivery result from several factors, including: (1) the nasal cavity has a relatively large surface area of about 150 cm² in man, (2) the submucosa of the lateral wall of the nasal cavity is richly supplied with vasculature, and (3) the nasal epithelium provides for a relatively high drug permeation capability due to thin single cellular layer absorption.

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used in the description of the invention and the appended claims, the singular forms “a,” “an” and “the” are intended to include the plural forms as well, unless the context clearly indicates otherwise.

I. Applications of the Present Invention.

The present invention finds use in research as well as veterinary and medical applications. Suitable subjects include both avians and mammals. The term “avian” as used herein includes, but is not limited to, chickens, ducks, geese, quail, turkeys and pheasants. The term “mammal” as used herein includes, but is not limited to, humans, non-human primates, cattle, sheep, goats, pigs, horses, cats, dog, rabbits, rodents (e.g., rats and/or mice), etc. In particular embodiments, the subject is a human subject that has been diagnosed with or is considered at risk for a metabolic disorder such as diabetes mellitus (e.g., type I or type II), metabolic syndrome, hyperglycemia, insulin resistance, glucose intolerance and/or obesity. The subject can further be a human subject that desires to lose weight for cosmetic and/or medical reasons. Alternatively, the subject can be a human subject that has been diagnosed with or is considered at risk for leptin resistance, gonadotropin deficiency, heart failure or ischemia, atherosclerosis, hypercholesterolemia, hypertension, amenorrhea, and/or polycystic ovary syndrome. Human subjects include neonates, infants, juveniles, and adults. In other embodiments, the subject used in the methods of the invention is an animal model of diabetes, hyperglycemia, metabolic syndrome, obesity, glucose intolerance, insulin resistance, leptin resistance, gonadotropin deficiency, heart failure or ischemia, atherosclerosis, hypercholesterolemia, hypertension, amenorrhea, and/or polycystic ovary syndrome.

In particular embodiments of the invention, the subject is a subject “in need of” the methods of the present invention, e.g., in need of the therapeutic effects of the inventive methods. For example, the subject can be a subject that has been diagnosed with or is considered at risk for diabetes mellitus (type I or type II), metabolic syndrome, hyperglycemia, insulin resistance, glucose intolerance, hyperphagia, obesity, leptin resistance, gonadotropin deficiency, heart failure or ischemia, atherosclerosis, hypercholesterolemia, hypertension, amenorrhea, and/or polycystic ovary syndrome, and the methods of the invention are practiced on the subject as a method of prophylactic or therapeutic treatment.

As used herein, the terms “delivery to,” “administration to” or “elevation of LC-COA in” the hypothalamus can refer to the hypothalamus when assessed as a whole, or can refer to particular regions of the hypothalamus (e.g., the mediobasal hypothalamus or the ARC).

As one aspect, the invention provides a method of elevating LC-CoA concentrations in the CNS, for example, the brain or the hypothalamus (e.g., the mediobasal hypothalamus including the ARC) of a subject by intranasally administering to the subject an effective amount of a compound or pharmaceutical composition that elevates LC-CoA levels in the CNS, for example, the brain or the hypothalamus (e.g., ARC). Methods of determining concentrations of LC-CoA are known, for example, by HPLC (see, e.g., Obici et al., (2003) Nature Medicine 9:756-761). In representative embodiments, hypothalamic (e.g., ARC) concentrations of LC-CoA are increased by about 25%, 40%, 50%, 75%, 100%, 200%, 250%, 300%, 350%, 400%, 500% or more.

The invention also provides a method of reducing glucose production in a subject by intranasally administering to the subject an effective amount of a compound or pharmaceutical composition that elevates LC-CoA levels in the CNS, for example, the brain or the hypothalamus (e.g., the ARC). The term “glucose production” can refer to whole animal glucose production or glucose production by particular organs or tissues (e.g., the liver and/or skeletal muscle). Glucose production can be determined by any method known in the art, e.g., by the pancreatic/insulin clamp technique. In representative embodiments, glucose production is reduced by at least about 20%, 25%, 40%, 50%, 75% or more. In particular embodiments, glucose production is normalized (e.g., as compared with a suitable healthy control) in the subject.

The invention further encompasses methods of treating diabetes (e.g., type-1 and/or type-2 diabetes), metabolic syndrome, hyperglycemia, insulin resistance and/or glucose intolerance in a subject by intranasally administering to the subject an effective amount of a compound or pharmaceutical composition that elevates LC-CoA levels in the CNS, for example, the brain or the hypothalamus (e.g., ARC).

As used herein, the term “diabetes” is used interchangeably with the term “diabetes mellitus.” The terms “diabetes” and “diabetes mellitus” are intended to encompass both insulin dependent and non-insulin dependent (type I and type II, respectively) diabetes mellitus, unless one condition or the other is specifically indicated. Methods of diagnosing diabetes are well known in the art. In humans, diabetes is typically characterized as a fasting level of blood glucose greater than or equal to about 140 mg/dl or as a plasma glucose level greater than or equal to about 200 mg/dl as assessed at about two hours following the oral administration of a glucose load of about 75 g. “Metabolic syndrome” is characterized by a group of metabolic risk factors in one person, including one or more of the following: central obesity (excessive fat tissue in and around the abdomen), atherogenic dyslipidemia (blood fat disorders—mainly high triglycerides and low HDL cholesterol—that foster plaque buildups in artery walls), raised blood pressure (e.g., 130/85 mmHg or higher), insulin resistance and/or glucose intolerance, a prothrombotic state (e.g., high fibrinogen or plasminogen activator inhibitor in the blood), and proinflammatory state (e.g., elevated high-sensitivity C-reactive protein in the blood). As used herein, the presence of metabolic syndrome in a subject can be diagnosed by any method currently known or later developed in the art. The criteria proposed by the Third Report of the National Cholesterol Education Program (NCEP) Expert Panel on Detection, Evaluation, and Treatment of High Blood Cholesterol in Adults (Adult Treatment Panel III) are the most widely used at this time to diagnose the metabolic syndrome. According to the ATP III criteria, the metabolic syndrome is identified by the presence of three or more of these components: central obesity as measured by waist circumference (men—greater than 40 inches; women—greater than 35 inches), fasting blood triglycerides greater than or equal to 150 mg/dL, blood HDL cholesterol (men—less than 40 mg/dl; women—less than 50 mg/dL), blood pressure greater than or equal to 130/85 mmHg, and fasting glucose greater than or equal to 110 mg/dL. The underlying causes of this syndrome are believed to be obesity, physical inactivity, and genetic factors. Subjects with metabolic syndrome are at increased risk of coronary heart disease, other diseases related to plaque buildup in artery walls (e.g., stroke and peripheral vascular disease) and/or type-2 diabetes. Metabolic syndrome has become increasingly common in the United States; as of October 2004, the American Heart Association estimates that about 47 million adults in the United States have metabolic syndrome.

Hyperglycemia is characterized by excessive blood (or plasma) glucose levels. Methods of diagnosing and evaluating hyperglycemia are known in the art. In general, fasting hyperglycemia is characterized by blood or plasma glucose concentration above the normal range after a subject has fasted for at least eight hours (e.g., the normal range is about 70-120 mg/dL). Postprandial hyperglycemia is generally characterized by blood or plasma glucose concentration above the normal range one to two hours after food intake by a subject.

By “insulin resistance” or “insulin insensitivity” it is meant a state in which a given level of insulin produces a less than normal biological effect (e.g., uptake of glucose). Insulin resistance is particularly prevalent in obese individuals or those with type-2 diabetes or metabolic syndrome. In type-2 diabetics, the pancreas is generally able to produce insulin, but there is an impairment in insulin action. As a result, hyperinsulinemia is commonly observed in insulin-resistant subjects. Insulin resistance is less common in type-I diabetics; although in some subjects, higher dosages of insulin have to be administered over time indicating the development of insulin resistance/insensitivity. The term “insulin resistance” or “insulin insensitivity” refers to whole animal insulin resistance/insensitivity unless specifically indicated otherwise. Methods of evaluating insulin resistance/insensitivity are known in the art, for example, hyperinsulinemic/euglycemic clamp studies, insulin tolerance tests, uptake of labeled glucose and/or incorporation into glycogen in response to insulin stimulation, and measurement of known components of the insulin signalling pathway.

“Glucose intolerance” is characterized by an impaired ability to maintain blood (or plasma) glucose concentrations following a glucose load (e.g., by ingestion or infusion) resulting in hyperglycemia. Glucose intolerance is generally indicative of an insulin deficiency or insulin resistance. Methods of evaluating glucose tolerance/intolerance are known in the art, e.g., the oral glucose tolerance test.

The invention further provides a method of improving hepatic autoregulation in a subject by intranasally administering to the subject an effective amount of a compound or pharmaceutical composition that elevates LC-COA levels in the CNS, for example, the brain or the hypothalamus (e.g., ARC). “Hepatic autoregulation” describes a phenomenon where, in the presence of basal insulin levels, increasing circulating levels of free fatty acid (e.g., by lipid infusion) stimulates gluconeogenesis, but does not alter endogenous glucose production via a compensatory decrease in hepatic glycogenolysis. This phenomenon can be dysfunctional, for example in diabetics, contributing to high plasma glucose levels. Improvement of hepatic autoregulation can be assessed by any method now known or later developed in the art (e.g., by increasing plasma free fatty acid concentrations and determining the extent of compensatory reduction in hepatic glycogenolysis and/or by measuring plasma glucose levels). In exemplary embodiments, the invention is practiced to achieve at least about a 10%, 20%, 30%, 40%, 50%, 75% or more improvement in hepatic autoregulation (for example, as determined by a corresponding decrease in blood or plasma glucose concentrations). In particular embodiments, hepatic autoregulation is returned to the normal range, e.g., as determined by comparison with a suitable healthy control.

As other aspects, the invention also encompasses methods of reducing appetite and/or food intake in a subject by intranasally administering to the subject an effective amount of a compound or pharmaceutical composition that elevates LC-COA levels in the CNS, for example, the brain or the hypothalamus (e.g., ARC). As used herein, the term “food” is intended to encompass both food for human consumption and animal feed. In particular embodiments, intake of food is reduced by at least about 5%, 10%, 15%, 20%, 25%, 50%, 60%, 70% or even more as compared with a suitable control or the subject's previous eating pattern or behavior. Reductions in food intake can be determined by any method now known or later developed by those skilled in the art, for example, by a reduction in caloric intake and/or a reduction in the frequency of eating. Likewise, reduction in appetite can be determined by any method now known or later developed in the art, e.g., as a decrease in the subjective sensation of hunger and/or reduction in food intake (as defined above).

As another illustrative embodiment, the invention further provides a method of treating obesity in a subject by intranasally administering to the subject an effective amount of a compound or pharmaceutical composition that elevates LC-CoA levels in the CNS, for example, the brain or the hypothalamus. Any degree of obesity can be treated, and the inventive methods can be practiced for research, cosmetic and/or medical purposes. In particular embodiments, the subject is at least about 5%, 10%, 20%, 30%, 50, 75% or even 100% or greater over normal body weight. Methods of determining normal body weight are known in the art. For example, in humans, normal body weight can be defined as a BMI index of 18.5-24.9 kg/meter² (NHLBI (National Heart Lung and Blood Institute) Obesity Education Initiative. The Practical Guide—Identification, Evaluation and Treatment of Overweight and Obesity in Adults. NIH Publication No. 004084 (2000); obtainable at http://www.nhIbi.nih.gov/guidelines/obesity/prctqdb.pdf). In particular embodiments, the invention is practiced to treat subjects having a BMI index of about 24.9 kg/meter² or greater. In representative embodiments, the methods of the invention result in at least about a 5%, 10%, 20%, 30%, 50% or greater reduction in degree of obesity (e.g., as determined by kg of weight loss or by reduction in BMI).

The invention can also be practiced to treat leptin resistance, gonadotropin deficiency, heart failure or ischemia, atherosclerosis, hypercholesterolemia, hypertension, amenorrhea, and/or polycystic ovary syndrome by intranasal administration of a compound or pharmaceutical composition that elevates LC-COA levels in the CNS, for example, the brain or the hypothalamus (e.g., the ARC).

As used herein, an “effective amount” refers to an amount of a compound or pharmaceutical composition that is sufficient to produce a desired effect, which is optionally a therapeutic effect (i.e., by administration of a therapeutically effective amount). For example, an “effective amount” can be an amount that is sufficient to elevate LC-COA in the CNS, for example, the brain or the hypothalamus (e.g., the ARC), to reduce glucose production, to reduce appetite and/or food intake, to improve hepatic autoregulation and/or to treat metabolic syndrome, hyperglycemia, glucose intolerance, insulin resistance, diabetes mellitus (e.g., type-2 or type-2 diabetes), obesity, leptin resistance, gonadotropin deficiency, heart failure or ischemia, atherosclerosis, hypercholesterolemia, hypertension, amenorrhea, and/or polycystic ovary syndrome.

A “therapeutically effective” amount as used herein is an amount that provides some improvement or benefit to the subject. Alternatively stated, a “therapeutically effective” amount is an amount that provides some alleviation, mitigation, delay and/or decrease in at least one clinical symptom and/or prevent the onset or progression of at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

The present invention can also be used to screen or identify compounds that can be administered intranasally to elevate LC-CoA in the CNS, for example, the brain or the hypothalamus (e.g., the ARC), to reduce glucose production, to reduce appetite and/or food intake, to improve hepatic autoregulation and/or to treat metabolic syndrome, hyperglycemia, glucose intolerance, insulin resistance, diabetes mellitus (e.g., type-1 or type-2 diabetes), obesity, leptin resistance, gonadotropin deficiency, heart failure or ischemia, atherosclerosis, hypercholesterolemia, hypertension, amenorrhea, and/or polycystic ovary syndrome. Subjects for use in the screening methods of the invention are as described above.

For example, in particular embodiments, a compound is delivered by intranasal administration to a subject and hypothalamic (e.g., ARC) levels of LC-CoAs are evaluated. An elevation in LC-CoA in the CNS, for example, the brain or the hypothalamus, indicates that the compound is a compound that can be administered intranasally to elevate LC-COA in the CNS, for example, the brain or the hypothalamus. Optionally, elevations in LC-COA are evaluated by comparison with a suitable control.

As another non-limiting example, the invention provides a method of identifying a compound that can be delivered by intranasal administration to a subject to reduce glucose production, improve hepatic autoregulation and/or to treat hyperglycemia, insulin resistance and/or glucose intolerance. In exemplary embodiments, a compound is administered intranasally to a subject and the levels of LC-CoAs in the CNS, for example, the brain or the hypothalamus (e.g., ARC) are determined. An elevation in LC-COA in the CNS (for example, the brain or the hypothalamus) indicates that the compound is a compound that can be administered intranasally to reduce glucose production, improve hepatic autoregulation and/or to treat hyperglycemia, insulin resistance and/or glucose intolerance. In particular embodiments, elevations in LC-COA are evaluated by comparison with a suitable control.

As a further non-limiting example, the invention provides a method of identifying a compound that can be delivered by intranasal administration to a subject to treat diabetes. In a representative embodiment, a compound is administered intranasally to a subject and the levels of LC-CoAs in the CNS, for example, the brain or the hypothalamus (e.g., ARC) are determined. An elevation in LC-COA in the CNS (for example, the brain or the hypothalamus) indicates that the compound is a compound that can be administered intranasally to treat diabetes. Optionally, elevations in LC-COA are evaluated by comparison with a suitable control.

The invention further provides a method of identifying a compound that can be delivered by intranasal administration to a subject to treat metabolic syndrome. In a representative embodiment, a compound is administered intranasally to a subject and the levels of LC-CoAs in the CNS, for example, the brain or the hypothalamus (e.g., ARC) are determined. An elevation in LC-CoA in the CNS (for example, the brain or the hypothalamus) indicates that the compound is a compound that can be administered intranasally to treat metabolic syndrome. Optionally, elevations in LC-COA are evaluated by comparison with a suitable control.

The invention further encompasses methods of identifying a compound that can be delivered by intranasal administration to a subject to reduce food intake and/or appetite. In a representative embodiment, a compound is administered intranasally to a subject and the levels of LC-CoAs in the CNS, for example, the brain or the hypothalamus (e.g., ARC) are determined. An elevation in LC-COA in the CNS (for example, the brain or the hypothalamus) indicates that the compound is a compound that can be administered intranasally to reduce food intake and/or appetite. Optionally, elevations in LC-COA are evaluated by comparison with a suitable control.

In yet other representative embodiments, the methods of the invention are practiced to identify a compound that can be delivered by intranasal administration to a subject to treat obesity. In a representative embodiment, a compound is administered intranasally to a subject and the levels of LC-CoAs in the CNS, for example, the brain or the hypothalamus (e.g., ARC) are determined. An elevation in LC-COA in the CNS, for example, the brain or the hypothalamus indicates that the compound is a compound that can be administered intranasally to treat obesity. Optionally, elevations in LC-COA are evaluated by comparison with a suitable control.

II. Compounds that Elevate LC-CoAs in the CNS.

The compositions and methods of the invention can be practiced with any compound that can be administered intranasally to elevate LC-COA in the CNS, for example, the brain or the hypothalamus (e.g., the ARC). For example, LC-COA levels can be elevated by reducing the activity of an LC-CoA-decreasing molecule in the CNS, for example, the brain or the hypothalamus (e.g., the ARC). Alternatively or additionally, LC-COA levels can be elevated by enhancing the activity of an LC-CoA-increasing molecule in the CNS, for example, the brain or the hypothalamus (e.g., the ARC).

As used herein, an LC-CoA-decreasing molecule is a molecule affecting lipid metabolism that has the effect of inhibiting production or promoting metabolism of LC-CoA. Included are enzymes or carrier proteins, now known or later discovered, that drive lipid metabolism away from production of LC-COA or toward metabolism of LC-COA. Suitable enzymes include, but are not limited to, enzymes that are involved in LC-COA metabolism. As non-limiting examples, the activity of the following enzymes and binding proteins can be reduced to decrease LC-COA levels in the CNS (e.g., hypothalamus): carnitine palmitoyl transferase 1 (CPT1, including the liver/hypothalamic isoform, CPT1L and the muscle isoform, CPT1 M), malonyl-CoA decarboxylase, carnitine acylcarnitine translocase, acyl-CoA dehydrogenase, 2-enoyl-CoA hydratase, 3-hydroxyacyl-CoA dehydrogenase, 3-oxoacyl-CoA thiolase, acyl-CoA hydrolase, fatty acyl-CoA oxidase, acyl-CoA binding protein, fatty acid synthase, gastric lipase, pancreatic lipase, non-pancreatic secretory phospholipase A2, non-pancreatic secretory phospholipase A3, pyruvate dehydrogenase kinase, acyl-CoA:cholesterol acyltransferase, AMP-protein kinase, 1-acyl-glycerol-3-phosphate acyltransferase 2, diacylglycerol acyltransferase, short chain acyl-CoA dehydrogenase, medium chain acyl-CoA dehydrogenase, long chain acyl-CoA dehydrogenase, monoamine oxidase, and microsomal triglyceride-transfer protein. Also encompassed are compounds that reduce or decrease the activity of any molecule that decreases the concentration of malonyl CoA in the CNS (e.g., malonyl decarboxylase), for example the brain or the hypothalamus (e.g., ARC).

The foregoing molecules can be pharmacologically modulated to decrease or increase LC-CoA levels in the CNS (e.g., hypothalamus). Thus, increasing the activity of a LC-CoA-decreasing molecule will decrease LC-CoA levels in the CNS. As described above, the activity of a LC-COA increasing molecule can be reduced to elevate LC-COA levels in the CNS.

As used herein, an LC-CoA-increasing molecule is a molecule affecting lipid metabolism that has the effect of promoting production and/or reducing metabolism of LC-CoA. Included are enzymes or carrier proteins, now known or later discovered, that drive lipid metabolism toward production of LC-COA or away from metabolism of LC-CoA. Enzymes include, but are not limited to, enzymes that directly produce LC-CoA. As non-limiting examples, the activity of the following enzymes and binding proteins can be increased to elevate LC-CoA levels in the CNS (e.g., hypothalamus): acetyl-CoA carboxylase, fatty acid transporter molecule and acyl-CoA synthetase.

The foregoing molecules can be pharmacologically modulated to decrease or increase LC-CoA levels in the CNS (e.g., hypothalamus). Thus, decreasing the activity of a LC-CoA-increasing molecule will decrease LC-CoA levels in the CNS. As described above, the activity of a LC-CoA increasing molecule can be increased to elevate LC-CoA levels in the CNS.

As used herein, “reducing [or decreasing] the activity” (or grammatical equivalents) of a molecule means either reducing the action (e.g., enzyme activity or binding to a ligand such as LC-COA) of the molecule as it relates to LC-COA production or metabolism and/or reducing the amount of such molecules (e.g., at the nucleic acid and/or protein level). It should be understood that the amount of the molecules can be reduced by increasing the rate of degradation or removal of the molecule and/or by decreasing the biosynthesis of the molecule. Conversely, “increasing [or enhancing] the activity” (or grammatical equivalents) of a molecule encompasses methods that increase the action of a molecule as it relates to LC-CoA production or metabolism and/or by increasing the amount of such molecules. The amount of a molecule can be increased by reducing the rate of degradation or removal of the molecule and/or increasing the biosynthesis of the molecule and/or by addition of the molecule (e.g., by administration of the molecule or by delivery of a nucleic acid encoding the molecule).

A. Compounds that Reduce the Activity of a LC-COA-Decreasing Molecule.

Examples of compounds that reduce or decrease the activity of a LC-CoA-decreasing molecule include small organic molecules, oligomers, polypeptides (including enzymes, antibodies and antibody fragments), carbohydrates, lipids, coenzymes, nucleic acids (including DNA, RNA and chimerics and analogues thereof), nucleic acid mimetics, nucleotides, nucleotide analogs, as well as other molecules (e.g., cytokines or enzyme inhibitors) that directly or indirectly inhibit molecules that promote production or accumulation of LC-CoA. In particular embodiments, the compound is an inhibitory nucleic acid such as an interfering RNA (RNAi) including short interfering RNAs (siRNA), an antisense nucleic acid, a ribozyme or a nucleic acid mimetic.

As used herein, a “small organic molecule” is an organic molecule of generally less than about 2000 MW that is not an oligomer. Small non-oligomeric organic compounds include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

Oligomers include oligopeptides, oligonucleotides, oligosaccharides, polylipids, polyesters, polyamides, polyurethanes, polyureas, polyethers, and poly (phosphorus derivatives), e.g. phosphates, phosphonates, phosphoramides, phosphonamides, phosphites, phosphinamides, etc., poly (sulfur derivatives) e.g., sulfones, sulfonates, sulfites, sulfonamides, sulfenamides, etc., where for the phosphorous and sulfur derivatives the indicated heteroatom are optionally bonded to C, H, N, O or S, and combinations thereof.

In particular embodiments, the compound is an antibody or antibody fragment that binds to a LC-CoA-decreasing molecule (e.g., an enzyme or binding protein) and reduces the activity thereof. The antibody or antibody fragment is not limited to any particular form and can be a polyclonal, monoclonal, bispecific, humanized, chimerized antibody or antibody fragment and can further be a Fab fragment, single chain antibody, and the like.

The nucleic acid sequences of numerous LC-CoA-decreasing molecules are known, which facilitates the synthesis of inhibitory oligonucleotides to reduce the activity of these molecules, see, e.g., carnitine palmitoyl transferase 1 (CPT1, including the liver/hypothalamic isoform, CPT1L (Genbank Accession No. NM_{—}001876) and the muscle isoform, CPT1 M (Genbank Accession No. NM_{—}004377); malonyl-CoA decarboxylase (Genbank Accession No. NM_{—}012213 [cytoplasmic and peroxisomal localization] and AF097832 [peroxisomal and mitochondrial localization]); carnitine acylcarnitine translocase (Genbank Accession Nos. NM_{—}000387); acyl-CoA dehydrogenase (Genbank Accession Nos. NM_{—}014384, NM 014049, NM_{—}000016, NM_{—}000018, NM_{—}000017, NM_{—}001609, NM_{—}001608); 2-enoyl-CoA hydratase (Genbank Accession No. NM_{—}004092); 3-hydroxyacyl-CoA dehydrogenase (Genbank Accession Nos. NM_{—}005327 [liver] and AF001903 [skeletal muscle]); fatty acid synthase (Genbank Accession No. BC063242); acyl-CoA binding protein (Accession No. BC029164); 3-oxoacyl-CoA thiolase (Accession No. NM_{—}001607); acyl-CoA hydrolase (Genbank Accession Nos. NM_{—}007274, NM_{—}181866, NM_{—}181865, NM_{—}181864, NM_{—}181863, NM_{—}181862), acyl-CoA oxidase (Genbank Accession Nos. NM_{—}003500, NM_{—}007292, NM_{—}004035, NM_{—}003501); pyruvate dehydrogenase kinase (Genbank Accession Nos. NM_{—}002610 [PDHK1], NM_{—}002611 [PDHK2], NM_{—}005391 [PDHK3] and NM_{—}002612 [PDHK4]); acyl-CoA:cholesterol acyltransferase (Genbank Accession Nos. NM_{—}003101 [SOAT1] and NM_{—}003578 [SOAT2]); AMP-protein kinase (GenBank Accession Nos. NM_{—}006251 [alpha 1 catalytic subunit], NM_{—}006252 [alpha 2 catalytic subunit], NM_{—}006253 [beta 1 non-catalytic subunit], NM_{—}002733 [gamma 1 non-catalytic subunit], NM_{—}016203 (gamma 2 non-catalytic subunit) and NM_{—}017431 [gamma 3 non-catalytic subunit]); 1-acyl-glycerol-3-phosphate acyltransferase 2 (GenBank Accession Nos. NM_{—}006412 [variant 1] an NM_{—}001012727 [variant 2]); diacylglycerol acyltransferase (GenBank Accession Nos. NM_{—}012079 [DGAT1] and NM_{—}032564 [DGAT2]); short chain acyl-CoA dehydrogenase (GenBank Accession No. NM_{—}000017); medium chain acyl-CoA dehydrogenase (GenBank Accession No. NM_{—}000016); long chain acyl-CoA dehydrogenase (GenBank Accession No. NM_{—}001599); monoamine oxidase (GenBank Accession Nos. NM_{—}000240 [MAOA] and NM_{—}000898 [MAOB]); and microsomal triglyceride-transfer protein (GenBank Accession No. NM_{—}000253).

Numerous compounds that reduce the activity of a LC-CoA-decreasing molecule are well known in the art and include but are not limited to inhibitors of the LC-CoA-decreasing molecules specifically listed herein, for example, inhibitors of CPT1 (including CPT1L and/or CPT1 M), malonyl-CoA decarboxylase, carnitine acylcarnitine translocase, acyl-CoA dehydrogenase, 2-enoyl-CoA hydratase, 3-hydroxyacyl-CoA dehydrogenase, 3-oxoacyl-CoA thiolase, acyl-CoA hydrolase, fatty acyl-CoA oxidase, acyl-CoA binding protein, gastric lipase, pancreatic lipase, non-pancreatic secretory phospholipase A2, non-pancreatic secretory phospholipase A3, fatty acid synthase, pyruvate dehydrogenase kinase, acyl-CoA:cholesterol acyltransferase, AMP-protein kinase, 1-acyl-glycerol-3-phosphate acyltransferase 2, diacylglycerol acyltransferase, short chain acyl-CoA dehydrogenase, medium chain acyl-CoA dehydrogenase, long chain acyl-CoA dehydrogenase, monoamine oxidase, and microsomal triglyceride-transfer protein.

Examples of compounds that can be used in the compositions and methods of the invention to reduce the activity of a LC-CoA-decreasing molecule include dichloroacetate and derivatives thereof, which are inhibitors of pyruvate dehydrogenase kinase (see, e.g., U.S. Pat. Nos. 5,643,951 and U.S. Pat. No. 4,558,050 to Stacpoole et al.); malonyl CoA decarboxylase inhibitors such as those described in U.S. patent Publications Nos. 2004/0082576, 2004/0092503, and 2004/0087627 (Arrhenius et al.), the cyanoamide compounds described in U.S. patent Publication Nos. 2005/0026945 (Kafka et al.), the piperidine compounds described in 2005/0032828 (Cheng et al.), the heterocyclic compounds described in U.S. patent Publication No. 2005/0026969 (Cheng et al.), and the cyanoguanidine-based azole compounds described in U.S. patent Publication No. 2005/0032824 (Cheng et al.); hydrazonopriopionic acid, which is an inhibitor of carnitine-acylcarnitine translocase (Rupp et al., (2002) Herz 27:621-636); carboxylesterase inhibitors, which are inhibitors of acyl-CoA hydrolase (Hosokawa et al., (2002) Arch. Biochem. Biophys. 389:245-253; fibrates such as (−)(3-trihalomethylphenoxy) (4-halophenyl) acetic acid derivatives (see, e.g., U.S. Pat. No. 6,624,194 to Luskey et al.); compounds able to activate PPARa and HNF4a, such as the carboxylic acids and their derivatives described in U.S. Pat. No. 6,303,653 to Bar-Tana and 3-thia fatty acids (Skorve et al., (1995) Xenobiotica 25:1181-1194); and fatty acid synthase inhibitors such as cerulenin and γ-substituted-α-methylene-β-carboxy-γ-butyrolactones as described by U.S. Pat. No. 5,981,575 to Kuhajda et al., and C75 (Kim et al., (2002) Am. J. Physiol. Endocrinol. Metab. 283:E867-E879; Gao et al., (2003) Proc. Nat'l Acad. Sci. 100:5628-5633; and Kumar et al., (2002) Proc. Nat'l Acad. Sci 99:1921-1925).

Additional compounds that decrease the activity of LC-CoA-decreasing molecules are shown in Table 1.

TABLE 1

| | Synonyms/Chemical | |
|---|---|---|
| Compound | Family/Type of Drug | Target |
| 447C88 | | ACAT inhibitor |
| 57-118 | | ACAT inhibitor |
| 58-035 | | ACAT inhibitor |
| Avasimibe | | ACAT Inhibitor |
| (GI-1011) | | |
| CI-999 | | ACAT inhibitor |
| CL-283546 | | ACAT inhibitor |
| CL-283796 | | ACAT inhibitor |
| CP-113818 | CP-105191 | ACAT inhibitor |
| E-5324 | | ACAT inhibitor |
| eflucimibe | F-12511 | ACAT inhibitor |
| | L0081 | |
| eldacimibe | ACA-147 | ACAT inhibitor |
| | WAY-125147 | |
| | WAY-ACA-147 | |
| F-1394 | | ACAT inhibitor |
| FCE-25390 | | ACAT inhibitor |
| FCE-28645A | | ACAT inhibitor |
| FR-129169 | | ACAT inhibitor |
| FR-186485 | FR-190809 | ACAT inhibitor |
| | FR-195249 | |
| K-10085 | | ACAT inhibitor |
| K-604 | | ACAT inhibitor |
| K-9406 | | ACAT inhibitor |
| KW-3033 | KF-20033 | ACAT inhibitor |
| KY-331 | | ACAT inhibitor |
| lecimitide | DuP-128 | ACAT inhibitor |
| LS-3115 | | ACAT inhibitor |
| malondiamides | | ACAT inhibitor |
| NTE-122 | | ACAT inhibitor |
| P-06139 | | ACAT inhibitor |
| pactimibe | CS-505 | ACAT inhibitor |
| PD-132301-2 | | ACAT inhibitor |
| RP 64477 | | ACAT inhibitor |
| RP 70676 | | ACAT inhibitor |
| RP 73163 | | ACAT inhibitor |
| SK&F-98016 | SR-12813 | ACAT inhibitor |
| SMP-797 | | ACAT inhibitor |
| TEI-6522 | | ACAT inhibitor |
| TEI-6620 | | ACAT inhibitor |
| TMP-153 | | ACAT inhibitor |
| U-73482 | U-84836 | ACAT inhibitor |
| U-76807 | | ACAT inhibitor |
| YM-750 | | ACAT inhibitor |
| melinamide | AC-223 | ACAT inhibitor |
| | Artes | Cholesterol antagonist |
| PD-13201-2 | | ACAT inhibitor |
| | | Cholesterol antagonist |
| YM-17E | | ACAT inhibitor |
| | | Cholesterol antagonist |
| crilvastatin | crilvastatine | ACAT inhibitor |
| | Cyclocor | Lipid peroxidase inhibitor |
| | cyclopide | |
| | cyclostatin | |
| | PMD-387 | |
| | riclostatin | |
| CEB-925 | | Cholesterol esterase inhibitor |
| | | ACAT inhibitor |
| CP-640186 | CP-610431 | ACC inhibitor |
| | CP-640188 | |
| Quizalofop | | ACC Inhibitor (plants) |
| clofibrate | fibrate analog | ACC inhibitor, likely through |
| | | activation of AMPK |
| gemfibrozil | fibrate analog | ACC inhibitor, likely through |
| | | activation of AMPK |
| CP-610431 | | ACC1 and ACC2 Inhibitor |
| Contracan | | Acyl CoA desaturase-1 inhibitor |
| XEN-103 | SCD1 inhibitors | Acyl CoA desaturase-1 inhibitor |
| CT32458 | | AGPAT2 Inhibitor |
| CT32615 | | AGPAT2 Inhibitor |
| AICAR | AMP analog | AMPK activator |
| (+)decanoyl-carnitine | (+)-acyl carnitines | CACT Inhibitor |
| Bupivacaine | Translocase Inhibitor | CACT Inhibitor |
| | (Thiophilic agent) | |
| Sulfobetaine | Translocase Inhibitor | CACT Inhibitor |
| | (Thiophilic agent) | |
| UK-5099 | Translocase Inhibitor | CACT Inhibitor |
| | (Thiophilic agent) | |
| MCHP | Derivatives of | CACT Inhibitor; MAO inhibitor |
| | Phenylethylhydrazine | |
| PPIB | Derivatives of | CACT Inhibitor; MAO inhibitor |
| | Phenylethylhydrazine | |
| 2-tetradecyl- | Glycidic Acids | CPT1 inhibition |
| glycidate (TDGA) | | |
| Amiodarone | | CPT1 inhibition |
| C75 | | FAS inhibition |
| Clomoxir | Glycidic Acids | CPT1 inhibition |
| Doxorubicin | | CPT1 inhibition |
| etomoxir | oxirane-carboxylate | CPT1 inhibition |
| Perhexiline | | CPT1 inhibition |
| Ranolazine | | CPT1 inhibition |
| Ro25-087 | oxamic acid | CPT1 inhibition |
| SDZ 265506 | acyl-tetrahedra intermediate | CPT1 inhibition |
| | analog | |
| SDZ 267597 | acyl-tetrahedra intermediate | CPT1 inhibition |
| | analog | |
| SDZ CPI975 | acyl-tetrahedra intermediate | CPT1 inhibition |
| | analog | |
| ST1326 | | CPT1 inhibition |
| Trimetazindine | | CPT1 inhibition |
| Hemipalmitoylcarnitinium | acyl-tetrahedra intermediate | CPT1, CPT2 inhibition |
| | analog | |
| (+)-octanoyl- | (+)-acyl carnitines | CPT1, CPT2, or CACT Inhibition |
| carnitine | | |
| (+)-palmitoyl- | (+)-acyl carnitines | CPT1, CPT2, or CACT Inhibition |
| carnitine | | |
| L-aminocarnitine | aminocarnitine derivative | CPT2 Inhibition |
| Amidepsine A | | DGAT Inhibitor |
| Xanthohumol | | DGAT Inhibitor |
| Cerulenin | | FAS Inhibitor |
| Thilactomycin | | FAS Inhibitor |
| Ebelactone A | | Gastric Lipase and Pancreatic |
| | | Lipase inhibitor |
| Orlistat | | Gastric Lipase and Pancreatic |
| | | Lipase inhibitor |
| CI-976 | PD-128042 | Inhibitor of liver and intestinal ACAT |
| Ro 23-9358 | | Inhibitor of non-pancreatic sPLA2 |
| BMS-181162 | | Inhibitor of non-pancreatic sPLA3 |
| Hypoglycin | toxin of ackee fruit | Inhibition of SCAD, MCAD, and |
| | | other acyl dehydrogenases |
| methylenecyclo- | metabolite of hypoglycin | Inhibition of SCAD, MCAD, and |
| propylacetic acid | | other acyl dehydrogenases |
| 3-mercapto- | | LCAD, MCAD Inhibitor |
| propionic acid | | |
| 8aR | | MTP Inhibitor |
| CP-346086 | | MTP Inhibitor |
| Implitapide | | MTP Inhibitor |
| (Bay 13-9952) | | |
| Dichloroacetate | | PDHK inhibitor |
| NVP-LAB121 | 3,3,3,-trifluoro-2-hydroxy-2- | PDHK inhibitor |
| | methlypropionamide | |
| NVP-LAB229 | 3,3,3,-trifluoro-2-hydroxy-2- | PDHK inhibitor |
| | methlypropionamide | |
| AZD7545 | | PDHK2 Inhibitor |

*Abbreviations:
ACAT: acyl-CoA:cholesterol acyltransferase
ACC: acetyl-CoA carboxylase
AICAR: 5-aminoimidazole-4-carboxamide 1-β-D-ribonucleoside
AMP: Adenosine mono-phosphate
AMPK: AMP-protein kinase
APGAT2: 1-acyl-glcerol-3-phosphate acyltransferase 2
CACT: carnitine acyl carnitine translocase
CPT: carnitine palmitoyl transferase
DGAT: diacylglycerol acyltransferase
FAS: fatty acid synthase
LCAD: Long chain acyl-CoA dehydrogenase
MAO: monoamine oxidase
MCAD: medium chain acyl-CoA dehydrogenase
MTP: microsomal triglyceride-transfer protein
PDHK: pyruvate dehydrogenase kinase
SCAD: short chain acyl-CoA dehydrogenase

In other embodiments of the invention, the compound comprises an inhibitory oligonucleotide, or a nucleic acid that encodes an inhibitory oligonucleotide, that specifically hybridizes to and reduces the activity of a LC-CoA-decreasing molecule, such as an enzyme. By “specifically hybridize” (or grammatical variations) it is meant that there is a sufficient degree of complementarity or precise pairing between the inhibitory oligonucleotide and the target nucleic acid such that stable and specific binding occurs between the oligonucleotide and the target. It is understood in the art that the sequence of the inhibitory oligonucleotide need not be 100% complementary to that of its target nucleic acid to be specifically hybridizable. An inhibitory oligonucleotide is specifically hybridizable when binding of the oligonucleotide to the target nucleic acid interferes with the normal function of the target nucleic acid (e.g., replication, transcription and/or translation), and there is a sufficient degree of complementarity to avoid non-specific binding of the inhibitory oligonucleotide to non-target nucleic acids under conditions in which specific binding is desired, e.g., under physiological conditions in the case of in vivo assays or therapeutic treatment and in the case of in vitro assays, under conditions in which the assays are performed. As is known in the art, a higher degree of sequence similarity is generally required for shorter oligonucleotides, whereas a greater degree of mismatched bases will be tolerated by longer oligonucleotides.

As discussed above, the nucleic acid sequences of a number of LC-CoA-increasing molecules are known in the art and can be used to readily design inhibitory oligonucleotides against a target of interest. Inhibitory oligonucleotides, or nucleic acids encoding the same, can be administered using any suitable method for nucleic acid delivery. Methods for delivering nucleic acids to a subject or target cell are well known in the art. The inhibitory oligonucleotide or nucleic acid encoding the inhibitory oligonucleotide can be incorporated into a delivery vector for administration, e.g., a viral or non-viral vector, including liposomal vectors and plasmids. Suitable viral vectors include adeno-associated virus, lentivirus and adenovirus vectors. The nucleic acid or vector typically includes transcriptional and translational control elements such as promoters, enhancers and terminators.

In particular embodiments, the compound comprises a ribozyme (or a nucleic acid that encodes a ribozyme) that reduces the activity of a LC-COA-decreasing molecule, such as an enzyme or binding protein. Ribozymes are RNA-protein complexes that cleave nucleic acids in a site-specific fashion. Ribozymes have specific catalytic domains that possess endonuclease activity (Kim et al., (1987) Proc. Natl. Acad. Sci. USA 84:8788; Gerlach et al., (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof, (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of nucleic acid expression may be particularly suited to therapeutic applications (Scanlon et al., (1991) Proc. Natl. Acad. Sci. USA 88:10591; Sarver et al., (1990) Science 247:1222; Sioud et al., (1992) J. Mol. Biol. 223:831).

As another approach, the compound can comprise an antisense oligonucleotide or a nucleic acid encoding an antisense oligonucleotide that is directed against the coding sequence for an LC-CoA-decreasing molecule, such as an enzyme or binding protein. The term “antisense oligonucleotide,” as used herein, refers to a nucleic acid that is complementary to and specifically hybridizes to a specified DNA or RNA sequence. Antisense oligonucleotides and nucleic acids that encode the same can be made in accordance with conventional techniques. See, e.g., U.S. Pat. No. 5,023,243 to Tullis; U.S. Pat. No. 5,149,797 to Pederson et al.

Those skilled in the art will appreciate that it is not necessary that the antisense oligonucleotide be fully complementary to the target sequence as long as the degree of sequence similarity is sufficient for the antisense nucleotide sequence to specifically hybridize to its target (as defined above) and reduce production of the enzyme (e.g., by at least about 30%, 40%, 50%, 60%, 70%, 80%, 90%, 95% or more).

To determine the specificity of hybridization, hybridization of such oligonucleotides to target sequences can be carried out under conditions of reduced stringency, medium stringency or even stringent conditions (e.g., conditions represented by a wash stringency of 3540% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively). See, e.g., Sambrook et al., Molecular Cloning, A Laboratory Manual (2d Ed. 1989) (Cold Spring Harbor Laboratory).

Alternatively stated, in particular embodiments, antisense oligonucleotides of the invention have at least about 60%, 70%, 80%, 90%, 95%, 97%, 98% or higher sequence similarity with the complement of the target sequence and reduces enzyme production (as defined above). In some embodiments, the antisense sequence contains 1, 2, 3, 4, 5, 6, 7, 8, 9 or 10 mismatches as compared with the target sequence.

As is known in the art, a number of different programs can be used to identify whether a nucleic acid or polypeptide has sequence similarity to a known sequence. Sequence similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nuc. Acid Res. 12, 387-395 (1984), or by inspection.

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which is described in Altschul et al., Methods in Enzymology, 266, 460-480 (1996) and available at http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., (1997) Nucleic Acids Res. 25, 3389-3402.

The length of the antisense oligonucleotide is not critical as long as it specifically hybridizes to the intended target and reduces enzyme production (as defined above) and can be determined in accordance with routine procedures. In general, the antisense oligonucleotide is from about eight, ten or twelve nucleotides in length and/or less than about 20, 30, 40, 50, 60, 70, 80, 100 or 150 nucleotides in length.

An antisense oligonucleotide can be constructed using chemical synthesis and enzymatic ligation reactions by procedures known in the art. For example, an antisense oligonucleotide can be chemically synthesized using naturally occurring nucleotides or various modified nucleotides designed to increase the biological stability of the molecules and/or to increase the physical stability of the duplex formed between the antisense and sense nucleotide sequences, e.g., phosphorothioate derivatives and acridine substituted nucleotides can be used.

Examples of modified nucleotides which can be used to generate the antisense oligonucleotide include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylam inomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotides of the invention further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described.

As another non-limiting example, one or all of the nucleotides in the oligonucleotide can contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87, 1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015.

The antisense oligonucleotide can be chemically modified (e.g., at the 3′ or 5′ end) to be covalently conjugated to another molecule. To illustrate, the antisense oligonucleotide can be conjugated to a molecule that facilitates delivery to a cell of interest, enhances absorption by the nasal mucosa (e.g, by conjugation to a lipophilic moiety such as a fatty acid), provides a detectable marker, increases the bioavailability of the oligonucleotide, increases the stability of the oligonucleotide, improves the formulation or pharmacokinetic characteristics, and the like. Examples of conjugated molecules include but are not limited to cholesterol, lipids, polyamines, polyamides, polyesters, intercalators, reporter molecules, biotin, dyes, polyethylene glycol, human serum albumin, an enzyme, an antibody or antibody fragment, or a ligand for a cellular receptor.

Other modifications to nucleic acids to improve the stability, nuclease-resistance, bioavailability, formulation characteristics and/or pharmacokinetic properties are known in the art.

RNA interference (RNAi) provides another approach for reducing the activity of a LC-CoA-decreasing molecule, such as an enzyme or binding protein. According to this embodiment, the compound comprises an RNAi molecule, a nucleic acid that encodes an RNAI molecule, or a nucleic acid that can be processed to produce an RNAi molecule. RNAi is a mechanism of post-transcriptional gene silencing in which double-stranded RNA (dsRNA) corresponding to a target sequence of interest is introduced into a cell or an organism, resulting in degradation of the corresponding mRNA. The mechanism by which RNAi achieves gene silencing has been reviewed in Sharp et al, (2001) Genes Dev 15: 485490; and Hammond et al., (2001) Nature Rev Gen 2:110-119). The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8).

Initial attempts to use RNAi in mammalian cells resulted in antiviral defense mechanisms involving PKR in response to the dsRNA molecules (see, e.g., Gil et al. (2000) Apoptosis 5:107). It has since been demonstrated that short synthetic dsRNA of about 21 nucleotides, known as “short interfering RNAs” (siRNA) can mediate silencing in mammalian cells without triggering the antiviral response (see, e.g., Elbashir et al., Nature (2001) 411:494-8; Caplen et al., (2001) Proc. Nat. Acad. Sci. 98:9742).

In one embodiment, RNAi molecules (including siRNA molecules) can be expressed from nucleic acid expression vectors in vitro or in vivo as short hairpin RNAs (shRNA; see Paddison et al., (2002), PNAS USA 99:1443-1448), which are believed to be processed in the cell by the action of the RNase Ill like enzyme Dicer into 20-25mer siRNA molecules. The shRNAs generally have a stem-loop structure in which two inverted repeat sequences are separated by a short spacer sequence that loops out. There have been reports of shRNAs with loops ranging from 3 to 23 nucleotides in length. The loop sequence is generally not critical. Exemplary loop sequences include the following motifs: AUG, CCC, UUCG, CCACC, CTCGAG, MGCUU, CCACACC and UUCMGAGA.

The RNAi can further comprise a circular molecule comprising sense and antisense regions with two loop regions on either side to form a “dumbbell” shaped structure upon dsRNA formation between the sense and antisense regions. This molecule can be processed in vitro or in vivo to release the dsRNA portion, e.g., a siRNA.

International patent publication WO 01/77350 describes a vector for bi-directional transcription to generate both sense and antisense transcripts of a heterologous sequence in a eukaryotic cell. This technique can be employed to produce RNAi for use according to the invention.

Shinagawa et al. (2003) Genes & Dev. 17:1340 reported a method of expressing long dsRNAs from a CMV promoter (a pol II promoter), which method is also applicable to tissue specific pol II promoters. Likewise, the approach of Xia et al., (2002) Nature Biotech. 20:1006, avoids poly(A) tailing and can be used in connection with tissue-specific promoters.

Methods of generating RNAi include chemical synthesis, in vitro transcription, digestion of long dsRNA by Dicer (in vitro or in vivo), expression in vivo from a delivery vector, and expression in vivo from a PCR-derived RNAi expression cassette (see, e.g., TechNotes 10(3) “Five Ways to Produce siRNAs,” from Ambion, Inc., Austin Tex.; available at www.ambion.com).

Guidelines for designing siRNA molecules are available (see e.g., literature from Ambion, Inc., Austin Tex.; available at www.ambion.com). In particular embodiments, the siRNA sequence has about 30-50% G/C content. Further, long stretches of greater than four T or A residues are generally avoided if RNA polymerase III is used to transcribe the RNA. Online siRNA target finders are available, e.g., from Ambion, Inc. (www.ambion.com), through the Whitehead Institute of Biomedical Research (www.jura.wi.mit.edu) or from Dharmacon Research, Inc. (www.dharmacon.com/).

The antisense region of the RNAi molecule can be completely complementary to the target sequence, but need not be as long as it specifically hybridizes to the target sequence (as defined above) and reduces production of the target enzyme (e.g., by at least about 30%, 40%, 50%, 60%, 70%, 80%, 90%, 95% or more). In some embodiments, hybridization of such oligonucleotides to target sequences can be carried out under conditions of reduced stringency, medium stringency or even stringent conditions, as defined above.

In other embodiments, the antisense region of the RNAi has at least about 60%, 70%, 80%, 90%, 95%, 97%, 98% or higher sequence similarity with the complement of the target sequence and reduces production of the target enzyme. In some embodiments, the antisense region contains 1, 2, 3, 4, 5, 6, 7, 8, 9 or 10 mismatches as compared with the target sequence. Mismatches are generally tolerated better at the ends of the dsRNA than in the center portion.

In particular embodiments, the RNAi is formed by intermolecular complexing between two separate sense and antisense molecules. The RNAi comprises a ds region formed by the intermolecular basepairing between the two separate strands. In other embodiments, the RNAi comprises a ds region formed by intramolecular basepairing within a single nucleic acid molecule comprising both sense and antisense regions, typically as an inverted repeat (e.g., a shRNA or other stem loop structure, or a circular RNAi molecule). The RNAi can further comprise a spacer region between the sense and antisense regions.

The RNAi molecule can contain modified sugars, nucleotides, backbone linkages and other modifications as described above for antisense oligonucleotides.

Generally, RNAi molecules are highly selective. If desired, those skilled in the art can readily eliminate candidate RNAi that are likely to interfere with expression of nucleic acids other than the target by searching relevant databases to identify RNAi sequences that do not have substantial sequence homology with other known sequences, for example, using BLAST (available at www.ncbi.nlm.nih.gov/BLAST).

Kits for the production of RNAi are commercially available, e.g., from New England Biolabs, Inc. and Ambion, Inc.

A nucleic acid mimetic is an artificial compound that behaves similarly to a nucleic acid by having the ability to base-pair with a complementary nucleic acid. Non-limiting examples of mimetics include peptide nucleic acids and phosphorothionate mimetics. Another example of a mimetic is an aptamer, which binds to and inhibits the target molecule in a manner similar to an antibody or small molecule inhibitor.

In embodiments of the invention, LC-COA levels in the CNS, for example, the brain or the hypothalamus are increased by reducing the activity of CPT1 (e.g., CPT1L). The compound can be a reversible or irreversible inhibitor of CPT1 activity and is optionally selective or specific for inhibition of CPT1L as compared with CPT1M so as to reduce side effects associated with inhibition of CPT1M.

The compound can be any compound as described above with respect to compounds that reduce the activity of LC-CoA-decreasing molecules.

In representative embodiments, the compound is an inhibitory nucleic acid that reduces the activity of CPT1 (e.g., CPT1L). Optionally, the compound is selective or even specific for CPT1L. The coding sequence of CPT1L is known in the art (see, e.g., Accession No. NM_{—}001876, CPT1A, human, liver; Britton et al. (1995) Proc. Natl. Acad. Sci. U.S.A. 92(6):1984-1988). One non-limiting example of a ribozyme for reducing the activity of human CPT1L comprises the sequence: 5′ACAGCACGCCGCUCUGAUGAGUCCGUAGAGGACGAAACCACGUUCUUCGUC-3′, where the bolded sequence is the catalytic core of a hammerhead enzyme. A nucleic acid comprising the ribozyme sequence can be administered to the subject; alternatively, a nucleic acid (e.g., a plasmid) that encodes the ribozyme can be administered.

In other embodiments, the compound is an organic molecule (e.g., a small organic molecule) that inhibits CPT1 (e.g., CPT1L) activity in the CNS, for example, the brain or the hypothalamus. Optionally, the compound is selective or even specific for CPT1L. Numerous compounds that inhibit CPT1 or CPT1L activity are known in the art. In general, such compounds include analogs of long-chain acylcarnitines (e.g., by modifying the ester bond), oxirane derivatives such as oxirane carboxylates, carnitine derivatives, aminocarnitine derivatives and acyl amino carnitine derivatives.

For example, compounds known to be CPT1 or CPT1L inhibitors encompass long chain alkyloxy- and aryloxy-substituted phosphinyloxy derivatives of carnitine, including long chain alkoxy- and aryloxy-substituted 3-carboxy-2-phosphinyloxy-1-propanaminium hydroxide inner salt derivatives (for example, SDZ-CPI-975), see, e.g., EP 0 574 355 B1 to Anderson et al.; and Deems et al., (1998) Am J. Physiol. 274 (Regulatory Integrative Comp. Physiol. 43): R524-528. In particular embodiments, as described in EP 0 574 355 B1, the compound has the formula of:
R₁—O—P(═X₁)(—X₂⁻)—O—CH(—CH₂—COOH)(—CH₂—N⁺R₂R₃R₄) formula (I)
where
X₁ and X₂ are independently O or S;
R₁ is R₅—Y—R₆— or R₇-Z-R₈— where
Y is —O—, —S—, —CH₂—, —CH═CH—, —C≡C—, —N(R₁₀)CO— or —CON(R₁₀)—;
Z is —O—, —S— or —CH₂—;
R₅ is straight or branched chain (C₁₋₁₇)alkyl or straight or branched chain ω-trifluoro-(C₁₋₈)alkyl;
R₆ is straight chained (C₂₋₁₈)alkylene; and the total number of carbon atoms in R₅—Y—R₆— is from 7 to 19;
R₇ is unsubstituted phenyl, phenoxyphenyl, biphenyl, naphthyl or naphthoxyphenyl; or phenyl, phenoxyphenyl, biphenyl, naphthyl or naphthoxyphenyl mono- or independently di- or independently trisubstituted with halogen, NO₂, NH₂, CN, (C₁₋₈)alkyl, (C₁₋₈)alkoxy, trifluoromethyl, trifluoromethoxy or acetyl;
R₈ is straight chained (C₃₋₁₅)alkylene, —(CH₂)ₘ—N(R₁₀)CO—(CH₂)ₙ—, —(CH₂)ₘ—CON(R₁₀)—(CH₂)ₙ— or —CH₂R₁₁OR₁₂—; where
m and n independently are 1 to 7;
R₁₀ is hydrogen, methyl, or ethyl;
R₁₁ is straight or branched chain (C1-7)alkylene;
R₁₂ is straight chained (C₂₋₇)alkylene; and
the total number of carbon atoms in the aryl substituents in R₇, and the total number of carbon atoms in R₈, not counting the significance of R₁₀, is from 3 to 15; and
R₂, R₃ and R4 are each independently straight or branched chain (C₁₋₄)alkyl;
in free acid form or in salt, physiologically hydrolysable ester or pro-drug form.

X₁ and X₂ preferably are both O or one of X₁ and X₂ preferably is O; X₁ and X₂ especially are both O. R₁ preferably is R₅—Y—R₆—. R₂, R₃ and R₄ preferably are methyl. Y preferably is —O— or —CH₂—, especially —CH₂—. Z preferably is O. R₅ preferably is straight chained, preferably straight chained (C₃₋₈)alkyl, especially hexyl. R₆ preferably is (C₃₋₈)alkylene, especially heptylene. When Y is —O— or —S—, the total number of carbon atoms in R₅—Y—R₆— preferably is from 11 to 17; when Y is —CH₂—, the total number of carbon atoms in R₅—Y—R₆— preferably is from 12 to 16; R₅—Y—R₆— especially is tetradecyl. R₇ is preferably substituted phenyl, phenoxyphenyl or naphthyl, it especially is optionally substituted phenyl. When it is substituted phenyl, it preferably is monosubstituted, particularly in the 4 position. R₈ preferably is straight chained alkylene, especially —(CH₂)₃₋₆—, particularly butylene. R₁₀ preferably is hydrogen or methyl. R₁₁ and ω-trifluoro-(C₁₋₈)alkyl preferably are straight chained. The total number of carbon atoms in R₈, not counting significance R₁₀, preferably is from 5 to 12.

Halogen is fluorine, chlorine, bromine or iodine, it preferably is fluorine or chlorine. (C₁₋₄)alkyl preferably is methyl. (C₁₋₈)alkoxy preferably is (C₁₋₆)alkoxy, it especially is hexyloxy.

Salts, e.g., metal salts such as the sodium or potassium salt and acid addition salts, such as the hydrochloride, can be formed using conventional methods, e.g., for acid addition salts, by reaction with an appropriate acid. Preferred salts are pharmacologically acceptable salts.

Physiologically hydrolysable esters include not only the esters formed with the carboxylic acid group of the carnitine moiety but also orthoesters formed with the phosphate moiety, e.g., the allyl ester.

The invention also includes pro-drug forms of the compounds of formula (I). Such pro-drugs are known and described in the literature, for example in PCT Publication WO 91/19721. These esters and pro-drugs include the pivaloyloxymethyl, 4-(2-methoxyphenoxy)-2-methylbutyryloxymethyl, N,N-dimethoxyethylcarbamoylmethyl, N-(3,6,9-trioxadecyl)-N-methylcarbamoylmethyl, N-(3,6,-dioxaheptyl)-N-methylcarbamoylmethyl, N,N-dipentylcarbamoylmethyl, N,N-dipropylcarbamoylmethyl, N,N-dibutylcarbamoylmethyl, and N-(2-methoxyphenoxyethyl)-N-methylcarbamoylmethyl esters of carnitine.

The compounds of formula (1) can exist in the form of optically active isomers and can be separated and recovered by conventional techniques. The L-carnitine forms of the compounds are preferred. Compounds in which one of X₁ or X₂ is a sulfur atom can exist in tautomeric form and in the form of diastereoisomers and can also be separated and recovered by conventional techniques. Similarly, compounds of the invention containing a double bond can exist in the form of geometric isomers, which can be readily separated and recovered by conventional procedures. Such isomeric forms are included in the scope of this invention.

A further subgroup of compounds of the invention is the compounds of formula (Iₛ)
R₁ₛ—O—P(═X₁)(—X²⁻)—O—CH(—CH₂—COOH)(—CH₂—N⁺(CH₃)₃) formula (Iₛ)
where
X₁ and X₂ are as defined above with respect to formula (I); and
R₁ is R₅—Yₛ—R₆— or R₇ₛ-Z-R₈ₛ— where
Yₛ is —O—, —CH₂—, —CH═CH—, —C—C—, —N(CH₃)CO—, —N(CH₂CH₃)CO— or —CON(CH₃)—;
Z, R₅ and R₆ are as defined above; and
the total number of carbon atoms in R₅—Yₛ—R₆— is from 7 to 19;
R₇ₛ is unsubstituted phenyl, phenoxyphenyl, naphthyl or naphthoxyphenyl; or
phenyl, phenoxyphenyl, naphthyl or naphthoxyphenyl mono- or independently di- or independently trisubstituted with fluorine, chlorine, NO₂, NH₂, CN, (C₁₋₆)alkyl, (C₁₋₆)alkoxy, trifluoromethyl, trifluoromethoxy or acetyl;
R₈ₛ is straight chained (C₃₋₁₂)alkylene, —(CH₂)ₘ—N(CH₃)CO—(CH₂)ₙ—, —(CH₂)ₘ—CON(CH₃)—(CH₂)ₙ— or —CH₂R₁₁ₛOR₁₂ₛ where
m and n are as defined above with respect to formula (I),
R₁₁ₛ is straight or branched chain (C₁₋₄)alkylene;
R₁₂ₛ is straight chained (C₂₋₅)alkylene; and the total number of carbon atoms in the aryl substituents in R₇ₛ and the total number of carbon atoms in R₈ₛ, not counting the methyl group attached to the nitrogen atom, is from 3 to 15;
in free acid form or in salt, or allyl, pivaloyloxymethyl or N,N-diethylcarboxamidylmethyl carboxylic ester or allyl phosphatidic orthoester form.

A further subgroup of compounds of the invention is the compounds of formula (Iₚ):
R₁ₚ—O—P(═X₁)(—X₂⁻)—O—CH(—CH₂—COOH)(—CH₂—N⁺R₂R₃R₄) formula (Ip)
where
X₁, X₂, R₂, R₃ and R₄ are as defined above with respect to formula (I), and
R₁ₚ is R₅ₚ—Yₚ—R₆ₚ— or R₇ₚ-Zₚ-R₈ₚ— where
Yₚ is —O—, —S—, —CH₂—, —CH═CH— or —C≡C—;
Zₚ is —O— or —S—;
R₅ₚ is straight or branched chain (C₁₋₇)alkyl;
R₆ₚ is straight chained (C₂₋₁₈)alkylene; and
the total number of carbon atoms in R₅ₚ—Yₚ—R₆ₚ— is from 7 to 19;
R₇ₚ is unsubstituted phenyl, biphenyl or naphthyl; or phenyl or naphthyl mono- or independently di- or independently trisubstituted with halogen, NO₂, (C₁₋₈)alkyl, (C₁₋₈)alkoxy, trifluoromethyl, trifluoromethoxy or acetyl;
R₈ₚ is straight chained (C₃₋₁₅)alkylene; and
the total number of carbon atoms in the substituents in R₇ₚ and in R₈ₚ is from 3to 15;
in free acid form or in pharmaceutically acceptable salt, physiologically hydrolysable ester or pro-drug form.

The compounds of formula (I), formula (Iₛ) and formula (Iₚ) can be made using methods known in the art, see e.g., EP 0 574 355 B1.

As another illustration, U.S. Pat. Nos. 6,444,701 and 6,369,073 to Giannessi et al. and Giannessi et al. (J. Med. Chem. 44:2383-2386 (2001)) disclose a large number of aminocarnitine derivatives that are inhibitors of CPT1. Accordingly, the compound can be an aminocarnitine derivative represented by the general formula:
X⁺—CH₂—CH(Z)-CH₂—Y formula (II)

wherein: X⁺ is N⁺(R₁,R₂,R₃),

wherein (R₁,R₂,R₃), being the same or different, are selected from the group consisting of hydrogen, a C₁-C₉ straight or branched alkyl group, —CH═NH(NH₂), —NH₂, and —OH; or one or more of R₁, R₂ and R₃, together with the nitrogen atom to which they are linked, form a saturated or unsaturated, monocyclic or bicyclic heterocyclic system; with the proviso that at least one of the R₁, R₂ and R₃ is different from hydrogen;

Z is selected from —OR₄, —OCOOR₄, —OCONHR₄, —OCSNHR₄, —OCSOR₄, —NHR₄, —NHCOR₄, —NHCSR₄, —NHCOOR₄, —NHCSOR₄, —NHCONHR₄, —NHCSNHR₄, —NHSOR₄, —NHSONHR₄, —NHSO₂R₄, —NHSO₂NHR₄, and —SR₄,

wherein —R₄ is a C₁-C₂₀ saturated or unsaturated, straight or branched alkyl group, optionally substituted with an A₁ group, wherein A₁ is selected from the group consisting of a halogen atom, or an aryl, heteroaryl, aryloxy or heteroaryloxy group, said aryl, heteroaryl, aryloxy or heteroaryloxy groups being optionally substituted with one or more C₁-C₂₀ saturated or unsaturated, straight or branched alkyl or alkoxy group and/or halogen atom;

Y is selected from the group consisting of —COO⁻, PO₃H⁻, —OPO₃H⁻, and tetrazolate-5-yl;

with the proviso that when Z is —NHCOR₄, Y is —COO⁻, then R₄ is C₂₀ alkyl;

with the proviso that when Z is —NHSO₂R₄, Y⁻ is —COO⁻, then R₄ is not tolyl;

with the proviso that when Z is —NHR₄, X⁺ is trimethylammonium and Y⁻ is —COO⁻, then R₄ is not C₁-C₆ alkyl, their (R,S) racemic mixtures, their single R or S enantiomers, or their pharmaceutically acceptable salts.

As examples of C₁-C₂₀ linear or branched alkyl group, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl and their possible isomers are meant, such as for example isopropyl, isobutyl, tert-butyl.

Examples of C₁-C₂₀ linear or branched alkenyl group are methylene, ethylidene, vinyl, allyl, propargyl, butylene, pentylene, wherein the carbon-carbon double bond, optionally in the presence of other carbon-carbon unsaturations, can be situated in the different possible positions of the alkyl chain, which can also be branched within the allowed isomery.

Examples of (C₆-C₁₄) aryl group are phenyl, 1- or 2-naphthyl, anthryl, optionally substituted as shown in the general definitions above-mentioned.

Examples of heterocyclic groups thienyl, quinolyl, pyridyl, N-methylpiperidinyl, 5-tetrazolyl, optionally substituted as shown in the general definitions above-mentioned.

Halogen atoms include fluorine, chlorine, bromine, iodine.

The compounds of formula (II) can also be in the form of inner salts.

In particular embodiments, the compounds comprise the compounds of formula (II) wherein N⁺(R₁,R₂,R₃) is trimethyl ammonium.

In other embodiments, the compounds comprise the compounds of formula (II) wherein two or more of R₁, R₂ and R₃, together with the nitrogen atom to which they are linked, form a saturated or unsaturated, monocyclic or bicyclic heterocyclic system; for example morpholinium, pyridinium, pyrrolidinium, quinolinium, quinuclidinium.

In further representative embodiments, the compounds comprise the compounds of formula (II) wherein R₁ and R₂ are hydrogen and R₃ is selected from the group consisting of —CH═NH(NH₂), —NH₂ and —OH.

Within particular embodiments of the present invention, the R₄ group can be a C₇-C₂₀ saturated or unsaturated, straight or branched alkyl group. In fact, it has been observed that a longer alkyl chain R₄ (>C10) can significantly increase the selectivity against CPT1. Examples of R₄ groups include heptyl, octyl, nonyl, decyl, undecyl, dodecyl, tridecyl, tetradecyl, pentadecyl, hexadecyl, heptadecyl, octadecyl, nonadecyl and eicosyl.

Examples of Z groups are ureido (—NHCONHR₄), and carbamate (—NHCOOR₄, —OCONHR₄) groups.

In particular embodiments, compounds of formula (II) comprise compounds wherein X⁺, R₁, R₂, R₃, have the above disclosed meanings, Z is ureido (—NHCONHR₄) or carbamate (—NHCOOR₄, —OCONHR₄), R₄ is a C₇-C₂₀, preferably a C₉-C₁₈ saturated or unsaturated, straight or branched alkyl group.

The compounds of formula (II) have an asymmetry center on the carbon atom bound to a Z group. For the purposes of the present invention, each compound of formula (II) can exist both as R,S racemic mixture and as separated R/S isomeric form.

The compounds of formula (II) are quaternary ammonium or phosphonium derivatives containing a Y⁻ anionic group. Dependent on pH, each compounds of formula (II) can exist indifferently as amphoion (inner salt) or as a compound wherein Y⁻ is present in the YH form. In such a case, X⁺ is salified with a pharmacologically acceptable acid. Formula (II) covers all these different possibilities.

In representative embodiments of the invention, the compound is R-4-trimethylammonium-3-(tetradecylcarbamoyl)-aminobutyrate (ST1326), R-4-trimethylammonium-3-(undecylcarbamoyl)-aminobutyrate (ST1327), R-4-trimethylammonium-3-(heptylcarbamoyl)-aminobutyrate (ST1328), S-4-trimethylammonium-3-(tetradecylcarbamoyl)-aminobutyrate (ST1340) and/or R-4-trimethylammonium-3-(dodecylcarbamoyl)am inobutyrate (ST1375).

Optionally, the methods of the invention comprise administration of a compound of formula (II) (e.g., ST1326) concurrently with mefformin therapy (see, e.g., PCT Publication WO 2004/069239 to Pessotto et al.). The compound of formula (II) and mefformin can be administered in the same or separate compositions. Further, mefformin can be administered intranasally or, alternatively, by peripheral routes including but not limited to intravenous or oral administration.

The compounds of formula (II) can be prepared by synthetic reactions that are well known in the art (see, e.g., U.S. Pat. Nos. 6,444,701 and 6,369,073 to Giannessi et al.).

The compound that inhibits CPT1 or CPT1L can alternatively be an aminocarnitine derivative as described by Giannessi et al. (J. Med. Chem. 46:303-309 (2003)) represented by the general formula:
(CH₃)₃N⁺CH₂CH(ZR)CH₂COO⁻ formula (III)
wherein:

Z=ureido, carbamate, sulfonamide, or sulfamide moieties; and

R═C₇ to C₁₄ linear alkyl chains.

The compounds of formula (III) include both R and S forms. In particular embodiments, the compound is the (R) form of the ureido derivative (ZR═NHCONHR, R═C₁₄), the sulfonamidic derivative (ZR═NHSO₂R, R=C₁₂), or the sulfamidic derivative (ZR═NHSO₂NHR, R═C₁₁).

Other compounds that are known to be CPT1 inhibitors include oxirane derivatives. Examples of oxirane derivatives include oxirane carboxylates such as methyl palmoxirate (Rupp et al., (2002) Herz 27:621-636), etomoxir and etomoxir derivatives, clomoxir, 2-(5-(4-chlorophenyl)pentyl)oxirane-2-carboxylate (POCA), and 2-tetradecylglycidate (TDGA) (see, e.g., Wolf, “Possible New Therapeutic Approach in Diabetes Mellitus by Inhibition of Carnitine Palmitoyltransferase 1 (CPT1), Pathogenesis and Management of Human Diabetes Mellitus, Workshop at the 23^{rd} Annual Meeting of the European Society for Clinical Investigation 1989, Athens, Greece; Hormone and Metabolic Research Supplement Series Volume No. 26); Ratheiser et al., (1991) Metabolism 40:1185-1190; and Anderson et al., (1995) J. Med. Chem 38:3448-3450; Anderson, (1998) Current Pharmaceutical Design 4:1-15); and U.S. patent Publication No. 2005/0004173 to Henkel et al. (arylalkyl- and aryloxyalkyl-substituted oxirane carboxylic acids). Oxirane carboxylic acids are also described in U.S. Pat. No. 6,479,676 to Wolf; U.S. Pat. No. 4,946,866 to Wolf; U.S. Pat. No. 4,430,339 to Eistetter et al.; U.S. Pat. No. 4,324,796 to Eistetter et al.; U.S. Pat. No. 4,788,306 to Schiehser et al.; U.S. Pat. No. 4,334,089 to Kraas et al.; U.S. Pat. No. 6,013,666 to Jew et al.; U.S. Pat. No. 5,739,159 to Wolf, and U.S. Pat. No. 4,788,304 to Marshall et al.

Other CPT1 or CPT1L inhibitors include but are not limited to 4-THA (2-hydroxy-3-propyl-4-[6-(tetrazol-5-yl)hexyloxy]acetophenone; Biochem. J. (1988) 252:409-414); 2-hydroxypropionic acid derivatives (U.S. Pat. No. 6,030,993 to Jew et al.), aminocarnitines and acylaminocarnitines (e.g., decanoyl-DL-amiocarnitine and palmitoyl-DL-aminocarnitine) as described by Jenkins et al., (1986) Proc. Natl. Acad. Sci 83:290-294), emeriamine (see, e.g., Kanamaru et al., Emeriamine: A new inhibitor of long chain fatty acid oxidation and its antidiabetic activity, Novel Microbial Products for Medicine and Agriculture, editors A. L. Demain et al., 135-144 (1989)), acylamidomorpholinium carnitine analogues (see, e.g., Savle et al.(1999) Bioorganic & Medicinal Chemistry Letters 9:3099-3102). Other compounds that inhibit CPT1 or CPT1L, including SDZ-269456 and SDZ-267-597, are described by Anderson, (1998) Current Pharmaceutical Design 4:1-15. Further examples of CPT1 or CPT1L inhibitors that can be administered according to the present invention include glibenclamide (Lehtihet et al., (2003) Am. J. Physiol. Endocrinol. Metabol. 185: E438446), S-15176, metoprolol, perhexiline, trimetazidine, oxfenicine, and amiodarone.(Rupp et al., (2002) Herz 27:621-636).

Additional compounds that inhibit CPT1, or more specifically CPT1L, are shown in Table 1 above.

B. Compounds that Enhance the Activity of a LC-COA-Increasing Molecule.

Compounds that increase or enhance the activity of a LC-CoA-increasing molecule are well known in the art and include but are not limited to compounds that activate, increase or enhance the activity of LC-COA-increasing molecules specifically listed herein, for example, acetyl-CoA carboxylase, fatty acid transporter molecule, and acyl-CoA synthetase.

Examples of compounds that increase or enhance the activity of a LC-CoA-increasing molecule include small organic molecules, oligomers, polypeptide (including enzymes, antibodies and antibody fragments), carbohydrates, lipids, coenzymes, nucleic acids (including DNA, RNA and chimerics and analogues thereof, nucleic acid mimetics, nucleotides, nucleotide analogs, as well as other molecules (e.g., cytokines or enzyme inhibitors) that directly or indirectly activate molecules that promote degradation of LC-COA.

The nucleic acid sequences of LC-CoA-increasing molecules are known, which facilitates the synthesis of nucleic acids encoding additional or modified copies of the molecules (or biologically active fragments) so as to increase the activity thereof, see, e.g., acetyl-CoA carboxylase (Genbank Accession No. AJ575592 [ACC2], AY315627 [ACC1], AY315626 [truncated ACC1 isoform]); fatty acid transporter molecule (Genbank Accession No. NM_{—}014031, NM_{—}198580, NM 005094, NM_{—}024330, NM_{—}003645, NM_{—}012254); and acyl-CoA synthetase (Genbank Accession No. NM 203380, NM_{—}203379, NM_{—}016234, NM_{—}203372, NM_{—}004457, NM_{—}001995, NM_{—}015256, NM_{—}022977, NM_{—}004458).

Another approach for increasing or enhancing the activity of a LC-COA-increasing molecule is to administer a nucleic acid encoding the molecule or a functional portion thereof. As discussed above, the nucleic acid sequences of a number of LC-CoA-increasing molecules are known in the art. The nucleic acid can be incorporated into a delivery vector for administration, e.g., a viral or non-viral vector, including liposomal vectors and plasmids. Suitable viral vectors include adeno-associated virus, lentivirus and adenovirus vectors. The nucleic acid or vector typically includes transcriptional and translational control elements such as promoters, enhancers and terminators. The nucleic acid can be administered to the subject, where the nucleic acid can be expressed to produce the LC-CoA-increasing molecule (e.g., in the CNS, for example, the brain or the hypothalamus (e.g., in the ARC).

III. Pharmaceutical Formulations and Modes of Intranasal Delivery.

The compounds to be administered according to the present invention encompass pharmaceutically acceptable salts of the compounds described above.

The term “pharmaceutically acceptable salts” refers to salts that retain the desired biological activity of the parent compound and do not impart undesired toxicological effects thereto.

Pharmaceutically acceptable base addition salts can be formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., (1977) “Pharmaceutical Salts,” J. of Pharma Sci. 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from the respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids including, for example, with inorganic acids, such as hydrochloric acid, hydrobromic acid, sulfuric acid or phosphoric acid; with organic acids such as carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as naturally-occurring alpha-amino acids, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine and iodine.

The compounds of the invention can be pro-drugs that are converted to the active compound (e.g., as described above) in vivo.

The compounds described above can further be modified to increase their lipophilicity and/or absorption across the nasal mucosa, e.g., by conjugation with lipophilic moieties such as fatty acids.

The invention also encompasses pharmaceutical compositions formulated for intranasal administration comprising one or more compounds that elevate intracellular LC-COA levels in the CNS (e.g., the brain or the hypothalamus (e.g., the ARC)) in a pharmaceutically acceptable carrier. The pharmaceutical composition can affect expression and/or activity of a LC-CoA-decreasing molecule and/or a LC-CoA-increasing molecule (each as described above). The one or more compounds can individually be prodrugs that are converted to the active compound in vivo. In particular embodiments, the invention provides a pharmaceutical composition formulated for intranasal administration comprising a compound that elevates intracellular LC-COA levels in the CNS, for example, the brain or the hypothalamus. Compounds that elevate intracellular LC-CoA levels are known in the art and are discussed in more detail hereinabove.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The formulations of the invention can optionally comprise medicinal agents, pharmaceutical agents, carriers, dispersing agents, diluents, humectants, wetting agents, thickening agents, odorants, humectants, penetration enhancers, preservatives, and the like.

The compositions of the invention can be formulated for intranasal administration in a pharmaceutical carrier in accordance with known techniques. See, e.g., Remington, The Science And Practice of Pharmacy (20^{th} edition, 2000). Suitable nontoxic pharmaceutically acceptable nasal carriers will be apparent to those skilled in the art of nasal pharmaceutical formulations (see, e.g., Remington's Pharmaceutical Sciences (Maack Publishing Co., Easton latest edition). Further, it will be understood by those skilled in the art that the choice of suitable carriers, absorption enhancers, humectants, adhesives, etc., will typically depend on the nature of the active compound and the particular nasal formulation, for example, a nasal solution (e.g., for use as drops, spray or aerosol), a nasal suspension, a nasal ointment, a nasal gel, or another nasal formulation.

The carrier can be a solid or a liquid, or both, and is optionally formulated with the composition as a unit-dose formulation. Such dosage forms can be powders, solutions, suspensions, emulsions and/or gels. With respect to solutions or suspensions, dosage forms can be comprised of micelles of lipophilic substances, liposomes (phospholipid vesicles/membranes), and/or a fatty acid (e.g., palmitic acid). In particular embodiments, the pharmaceutical composition is a solution or suspension that is capable of dissolving in the fluid secreted by mucous membranes of the olfactory epithelium, which can advantageously enhance absorption.

The pharmaceutical composition can be an aqueous solution, a nonaqueous solution or a combination of an aqueous and nonaqueous solution.

Suitable aqueous solutions include but are not limited to aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include but are not limited to nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by the mucosal membranes of the nasal cavity.

Examples of powder formulations include without limitation simple powder mixtures, micronized powders, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the compound is one that is at least partially, or even substantially (e.g., at least 80%, 90%, 95% or more) soluble in the fluids that are secreted by the nasal mucosa (e.g., the mucosal membranes that surround the cilia of the olfactory receptor cells of the olfactory epithelium) so as to facilitate absorption. Alternatively or additionally, the compound can be formulated with a carrier and/or other substances that foster dissolution of the agent within nasal secretions, including without limitation fatty acids (e.g., palmitic acid), gangliosides (e.g., GM-I), phospholipids (e.g., phosphatidylserine, and emulsifiers (e.g., polysorbate 80).

Optionally, drug solubilizers can be included in the pharmaceutical composition to improve the solubility of the compound and/or to reduce the likelihood of disruption of nasal membranes which can be caused by application of other substances, for example, lipophilic odorants. Suitable solubilizers include but are not limited to amorphous mixtures of cyclodextrin derivatives such as hydroxypropylcylodextrins (see, for example, Pitha et al., (1988) Life Sciences 43:493-502.

In representative embodiments, the compound is lipophilic to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-I), phospholipids (e.g., phosphatidylserine), and emulsifiers (e.g., polysorbate 80), bile salts such as sodium deoxycholate, and detergent-like substances including, for example, polysorbate 80 such as Tween, octoxynol such as Triton™ X-100, and sodium tauro-24,25-dihydrofusidate (STDHF). See Lee et al., Biopharm., April 1988 issue:3037.

In particular embodiments of the invention, the active compound is combined with micelles comprised of lipophilic substances. Such micelles can modify the permeability of the nasal membrane to enhance absorption of the compound. Suitable lipophilic micelles include without limitation gangliosides (e.g., GM-1 ganglioside), and phospholipids (e.g., phosphatidylserine). Bile salts and their derivatives and detergent-like substances can also be included in the micelle formulation. The active compound can be combined with one or several types of micelles, and can further be contained within the micelles or associated with their surface.

Alternatively, the active compound can be combined with liposomes (lipid vesicles) to enhance absorption. The active compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

In representative embodiments, the pH of the pharmaceutical composition ranges from about 2, 3, 3.5 or 5 to about 7, 8 or 10. Exemplary pH ranges include without limitation from about 2 to 8, from about 3.5 to 7, and from about 5 to 7. Those skilled in the art will appreciate that because the volume of the pharmaceutical composition administered is generally small, nasal secretions may alter the pH of the administered dose, since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include but are not limited to acetate, citrate, prolamine, carbonate and phosphate buffers.

In embodiments of the invention, the pH of the pharmaceutical composition is selected so that the internal environment of the nasal cavity after administration is on the acidic to neutral side, which (1) can provide the active compound in an un-ionized form for absorption, (2) prevents growth of pathogenic bacteria in the nasal passage that is more likely to occur in an alkaline environment, and (3) reduces the likelihood of irritation of the nasal mucosa.

Further, in particular embodiments, the net charge on the compound is a positive or neutral charge.

According to other embodiments of the invention, the compound has a molecular weight of about 50 kilodaltons, 10 kilodaltons, 5 kilodaltons, 2 kilodaltons, 1 kilodalton, 500 daltons or less.

For liquid and powder sprays or aerosols, the pharmaceutical composition can be formulated to have any suitable and desired particle size. In illustrative embodiments, the majority and/or the mean size of the particles or droplets range in size from greater than about 1, 2.5, 5, 10 or 15 microns and/or less than about 25, 30, 40, 50, 60 or 75 microns. Representative examples of suitable ranges for the majority and/or mean particle or droplet size include, without limitation, from about 5 to 50 microns, from about 20 to 40 microns, and from about 15 to 30 microns, which facilitate the deposition of an effective amount of the active compound in the nasal cavity (e.g., in the olfactory region and/or in the sinus region). In general, particles or droplets smaller than about 5 microns will be deposited in the trachea or even the lung, whereas particles or droplets that are about 50 microns or larger generally do not reach the nasal cavity and are deposited in the anterior nose.

In particular embodiments, the pharmaceutical composition is isotonic to slightly hypertonic, e.g., having an osmolarity ranging from about 150 to 550 mOsM. As another particular example, the pharmaceutical composition is isotonic having, e.g., an osmolarity ranging from approximately 150 to 350 mOsM.

According to particular methods of intranasal delivery, it can be desirable to prolong the residence time of the pharmaceutical composition in the nasal cavity (e.g., in the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the dosage formulation can also provide a means of prolonging contact of agent with nasal epithelium. The pharmaceutical composition can be formulated as a nasal emulsion, ointment or gel, which offer advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of 2% by weight or less.

The pharmaceutical composition can optionally contain an odorant, e.g., as described in EP 0 504 263 B1 to provide a sensation of odor, to aid in inhalation of the composition so as to promote delivery to the olfactory epithelium and/or to trigger transport by the olfactory neurons.

As another option, the composition can comprise a flavoring agent, e.g., to enhance the taste and/or acceptability of the composition to the subject.

The invention also encompasses methods of intranasal administration of the pharmaceutical formulations of the invention. In particular embodiments, the pharmaceutical composition is delivered to the olfactory region and/or the sinus region of the nose. The olfactory region is a small area located in the upper third of the nasal cavity for deposition and absorption by the olfactory epithelium and subsequent transport by olfactory receptor neurons. Located on the roof of the nasal cavity, the olfactory region is desirable for delivery because it is the only known part of the body in which an extension of the CNS comes into contact with the environment (Bois et al., Fundamentals of Otolaryngology, p. 184, W.B. Saunders Co., Phila., 1989).

In particular embodiments, the pharmaceutical composition is administered to the subject in an effective amount, optionally, a therapeutically effective amount (each as described hereinabove). Dosages of pharmaceutically active compositions can be determined by methods known in the art, see, e.g., Remington's Pharmaceutical Sciences (Maack Publishing Co., Easton, Pa.; 18^{th} edition, 1990).

A therapeutically effective amount will vary with the age and general condition of the subject, the severity of the condition being treated, the particular compound or composition being administered, the duration of the treatment, the nature of any concurrent treatment, the carrier used, and like factors within the knowledge and expertise of those skilled in the art. As appropriate, a therapeutically effective amount in any individual case can be determined by one of ordinary skill in the art by reference to the pertinent texts and literature and/or by using routine experimentation (see, e.g., Remington, The Science and Practice of Pharmacy (20^{th} ed. 2000)).

As a general proposition, a dosage from about 0.01 or 0.1 to about 1, 5, 10, 20, 50, 75, 100, 150, 200 or 250 mg/kg body weight will have therapeutic efficacy, with all weights being calculated based upon the weight of the active ingredient, including salts.

The pharmaceutical composition can be delivered in any suitable volume of administration. In representative embodiments of the invention, the administration volume for intranasal delivery ranges from about 25 microliters to 200 microliters or from about 50 to 150 microliters. In particular embodiments, the administration volume is selected to be small enough to allow for the dissolution of an effective amount of the active compound but sufficiently large to prevent therapeutically significant amounts of inhibitor from escaping from the anterior chamber of the nose and/or draining into the throat, post nasally.

Any suitable method of intranasal delivery can be employed for delivery of the pharmaceutical compound. To illustrate, the pharmaceutical composition can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by drops, spray or aerosol.

In representative embodiments, the pharmaceutical formulation is directed upward during administration, to enhance delivery to the upper third (e.g., the olfactory region) and the side walls (e.g., nasal epithelium) of the nasal cavity.

The methods of intranasal delivery can be carried out once or multiple times, and can further be carried out daily, every other day, etc., with a single administration or multiple administrations per day of administration, (e.g., 2, 3, 4 or more times per day of administration). In other embodiments, the methods of the invention can be carried out on an as-needed by self-medication.

Further, the pharmaceutical compositions of the present invention can optionally be administered in conjunction with other therapeutic agents, for example, other therapeutic agents useful in the treatment of hyperglycemia, diabetes, metabolic syndrome and/or obesity. For example, the compounds of the invention can be administered in conjunction with insulin therapy and/or hypoglycemic agents (e.g., mefformin). The additional therapeutic agent(s) can be administered concurrently with the compounds of the invention, in the same or different formulations. As used herein, the word “concurrently” means sufficiently close in time to produce a combined effect (that is, concurrently can be simultaneously, or it can be two or more events occurring within a short time period before or after each other).

The foregoing is illustrative of the present invention, and is not to be construed as limiting thereof. The invention is defined by the following claims, with equivalents of the claims to be included therein.

We claim:

1. A pharmaceutical composition formulated for intranasal administration comprising a compound that elevates long-chain acyl-CoA (LC-CoA) levels in the hypothalamus in a pharmaceutically acceptable carrier.

2. The pharmaceutical composition of claim 1, wherein the pharmaceutical composition is formulated for intranasal administration to the olfactory region and/or sinus region.

3. The pharmaceutical composition of claim 1, wherein the compound elevates LC-COA levels in the arcuate nucleus of the hypothalamus.

4. The pharmaceutical composition of claim 1, wherein the composition is an aqueous solution.

5. The pharmaceutical composition of claim 4, wherein the aqueous solution is selected from the group consisting of an aqueous gel, an aqueous suspension, an aqueous microsphere suspension, an aqueous microsphere dispersion, an aqueous liposomal dispersion, aqueous micelles of liposomes, an aqueous microemulsion, and any combination of the foregoing.

6. The pharmaceutical composition of claim 1, wherein the composition is a nonaqueous solution.

7. The pharmaceutical composition of claim 6, wherein the nonaqueous solution is selected from the group consisting of a nonaqueous gel, a nonaqueous suspension, a nonaqueous microsphere suspension, a nonaqueous microsphere dispersion, a nonaqueous liposomal dispersion, a nonaqueous emulsion, a nonaqueous microemulsion, and any combination of the foregoing.

8. The pharmaceutical composition of claim 1, wherein the composition is a powder formulation.

9. The pharmaceutical composition of claim 8, wherein the powder formulation is selected from the group consisting of a simple powder mixture, a micronized powder, powder microspheres, coated powder microspheres, and any combination of the foregoing.

10. The pharmaceutical composition according to claim 1, wherein the pharmaceutical composition has a pH in the range of pH 3.5 to pH 7.

11. The pharmaceutical composition of claim 1, wherein the osmolarity of the composition is in the range of 150 to 550 mOsM.

12. The pharmaceutical composition of claim 11, wherein the osmolarity of the composition is in the range of 150 to 350 mOsM.

13. The pharmaceutical composition of claim 1, wherein the composition is in the form of liquid droplets or solid particles.

14. The pharmaceutical composition of claim 13, wherein the majority and/or mean size of the liquid droplets or solid particles range in size from 5 microns to 50 microns.

15. The pharmaceutical composition of claim 14, wherein the majority and/or mean size of the liquid droplets or solid particles range in size from 10 microns to 40 microns.

16. The pharmaceutical composition of claim 1, wherein the composition is in the form of a nasal spray, nasal drops or an aerosol.

17. The pharmaceutical composition of claim 1, wherein the compound has a molecular weight of 50,000 daltons or less.

18. The pharmaceutical composition of claim 1, wherein the composition comprises at least one absorption enhancer.

19. The pharmaceutical composition of claim 18, wherein the absorption enhancer comprises a chelating agent or a fatty acid.

20. The pharmaceutical composition of claim 1, wherein the composition comprises a compound that reduces the activity of an enzyme or binding protein selected from the group consisting of carnitine palmitoyl transferase 1 (CPT1), malonyl-CoA decarboxylase, carnitine acylcarnitine translocase, acyl-CoA dehydrogenase, 2-enoyl-CoA hydratase, 3-hydroxyacyl-CoA dehydrogenase, 3-oxoacyl-CoA thiolase, acyl-CoA hydrolase, fatty acyl-CoA oxidase, acyl-CoA binding protein, fatty acid synthase, gastric lipase, pancreatic lipase, non-pancreatic secretory phospholipase A2, non-pancreatic secretory phospholipase A3, pyruvate dehydrogenase kinase, acyl-CoA:cholesterol acyltransferase, 5′-AMP-protein kinase, 1-acyl-glycerol-3-phosphate acyltransferase 2, diacylglycerol acyltransferase, short chain acyl-CoA dehydrogenase, medium chain acyl-CoA dehydrogenase, long chain acyl-CoA dehydrogenase, monoamine oxidase, and microsomal triglyceride-transfer protein.

21. The pharmaceutical composition of claim 20, wherein the composition comprises a fibrate or a pharmaceutically acceptable salt thereof.

22. The pharmaceutical composition of claim 21, wherein the composition comprises a (−)(3-trihalomethylphenoxy)(4-halophenyl)acetic acid derivative or a pharmaceutically acceptable salt thereof.

23. The pharmaceutical composition of claim 22, wherein the composition comprises hydrazonopriopionic acid or a pharmaceutically acceptable salt thereof.

24. The pharmaceutical composition of claim 20, wherein the composition comprises a 3-thia fatty acid or a pharmaceutically-acceptable salt thereof.

25. The pharmaceutical composition of claim 20, wherein the composition comprises a carboxylesterase inhibitor or a pharmaceutically-acceptable salt thereof.

26. The pharmaceutical composition of claim 20, wherein the composition comprises a compound selected from the group consisting of cerulenin, C75, a γ-substituted-α-methylene-β-carboxy-γ-butyrolactone, and a pharmaceutically acceptable salt of any of the foregoing.

27. The pharmaceutical composition of claim 20, wherein the composition comprises dichloroacetate, a dichloroacetate derivative, or a pharmaceutically acceptable salt of any of the foregoing.

28. The pharmaceutical composition of claim 20, wherein the composition comprises an inhibitory nucleic acid selected from the group consisting of an antisense RNA, an interfering RNA (RNAi), an aptamer, and a ribozyme.

29. The pharmaceutical composition of claim 20, wherein the composition comprises a nucleic acid selected from the group consisting of a nucleic acid that encodes an antisense RNA, a nucleic acid that encodes an RNAi, a nucleic acid that encodes an aptamer, and a nucleic acid that encodes a ribozyme.

30. The pharmaceutical composition of claim 20, wherein the composition comprises a compound that reduces the activity of a CPT1.

31. The pharmaceutical composition of claim 30, wherein the composition comprises a compound that reduces the activity of a liver isoform of CPT1 (CPT1L).

32. The pharmaceutical composition of claim 31, wherein the compound is selective for CPT1L as compared with the muscle isoform of CPT1 (CPT1M).

33. The pharmaceutical composition of claim 30, wherein the CPT1 inhibitor is selected from the group consisting of an oxirane derivative, a carnitine derivative, an aminocarnitine derivative, an acyl aminocarnitine derivative, compounds that are analogs of long-chain acylcarnitines, and pharmaceutically acceptable salts of any of the foregoing.

34. The pharmaceutical composition of claim 33, wherein the compound is an oxirane carboxylate or a pharmaceutically acceptable salt thereof.

35. The pharmaceutical composition of claim 34, wherein the oxirane derivative is selected from the group consisting of etomoxir, an etomoxir derivative, clomoxir, POCA, 2-tetradecylglycidate (TDGA), methyl palmoxirate, and a pharmaceutically acceptable salt thereof.

36. The pharmaceutical composition of claim 33, wherein the carnitine derivative is a long chain alkoxy- or aryloxy-substituted phosphinyloxy carnitine derivative.

37. The pharmaceutical formulation of claim 36, wherein the carnitine derivative is SDZ-CPI-975 or a pharmaceutically acceptable salt thereof.

38. The pharmaceutical composition of claim 33, wherein the carnitine derivative is an acylamidomorpholinium carnitine analog.

39. The pharmaceutical composition of claim 33, wherein the aminocarnitine derivative is selected from the group consisting of R4-trimethylammonium-3-[tetradecylcarbamoyl)-aminobutyrate (ST1326), R4-trimethylammonium-3-(undecylcarbamoyl)-aminobutyrate (ST1327), R4-trimethylammonium-3-(heptylcarbamoyl)-aminobutyrate (ST1328), S4-trimethylammonium-3-(tetradecylcarbamoyl)-aminobutyrate (ST1340), R4-trimethylammonium-3-(dodecylcarbamoyl)aminobutyrate (ST1375), and a pharmaceutically acceptable salt of any of the foregoing.

40. The pharmaceutical composition of claim 30, wherein the composition comprises a compound selected from the group consisting of glibenclamide, 4-THA, a 2-hydroxypropionic acid derivative, S-15176, metoprolol, perhexiline, trimetazidine, oxfenicine, amiodarone, and a pharmaceutically-acceptable salt of any of the foregoing.

41. The pharmaceutical composition of claim 1, wherein the composition comprises a compound that enhances the activity of an enzyme or binding protein selected from the group consisting of acetyl-CoA carboxylase, fatty acid transporter molecule and acyl-CoA synthetase.

42. A method of elevating LC-COA levels in the hypothalamus of a mammalian subject comprising intranasally administering to the mammalian subject an effective amount of a pharmaceutical composition according to claim 1.

43. The method of claim 42, wherein the pharmaceutical composition is administered to the olfactory and/or sinus region.

44. The method of claim 42, wherein LC-CoA levels are elevated in the arcuate nucleus of the hypothalamus.

45. The method of claim 42, wherein the subject is a human subject.

46. The method of claim 42, wherein the subject is an animal model of diabetes mellitus, metabolic syndrome and/or obesity.

47. The method of claim 42, wherein the subject has diabetes mellitus.

48. The method of claim 42, wherein the subject has metabolic syndrome.

49. The method of claim 42, wherein the subject is at least 20% over normal body weight.

50. The method of claim 42, wherein the pharmaceutical composition is in the form of nasal drops, a nasal spray or an aerosol.

51. A method of treating diabetes mellitus in a mammalian subject comprising intranasally administering to the mammalian subject an effective amount of a pharmaceutical composition according to claim 1.

52. A method of treating metabolic syndrome in a mammalian subject comprising intranasally administering to the mammalian subject an effective amount of a pharmaceutical composition according to claim 1.

53. A method of improving hepatic autoregulation in a mammalian subject comprising intranasally administering to the mammalian subject an effective amount of a pharmaceutical composition according to claim 1.

54. A method of reducing glucose production in a mammalian subject comprising intranasally administering to the mammalian subject an effective amount of a pharmaceutical composition according to claim 1.

55. A method of reducing food intake in a mammalian subject comprising intranasally administering to the mammalian subject an effective amount of a pharmaceutical composition according to claim 1.

56. A method of treating obesity in a mammalian subject comprising intranasally administering to the mammalian subject an effective amount of a pharmaceutical composition according to claim 1.

57. A method of elevating LC-COA levels in the hypothalamus of a mammalian subject comprising intranasally administering to the mammalian subject an effective amount of a pharmaceutical composition according to claim 30.

58. A method of treating diabetes mellitus in a mammalian subject comprising intranasally administering to the mammalian subject an effective amount of a pharmaceutical composition according to claim 30.

59. A method of treating metabolic syndrome in a mammalian subject comprising intranasally administering to the mammalian subject an effective amount of a pharmaceutical composition according to claim 30.

60. A method of improving hepatic autoregulation in a mammalian subject comprising intranasally administering to the mammalian subject an effective amount of a pharmaceutical composition according to claim 30.

61. A method of reducing glucose production in a mammalian subject comprising intranasally administering to the mammalian subject an effective amount of a pharmaceutical composition according to claim 30.

62. A method of reducing food intake in a mammalian subject comprising intranasally administering to the mammalian subject an effective amount of a pharmaceutical composition according to claim 30.

63. A method of treating obesity in a mammalian subject comprising intranasally administering to the mammalian subject an effective amount of a pharmaceutical composition according to claim 30.

64. A method of elevating LC-COA levels in the hypothalamus of a mammalian subject comprising intranasally administering to the mammalian subject an effective amount of a compound that elevates long-chain acyl-CoA (LC-CoA) levels in the hypothalamus.

65. The method of claim 64, wherein the compound reduces the activity of a CPT1.

66. A method of treating diabetes mellitus in a mammalian subject comprising intranasally administering to the mammalian subject an effective amount of a compound that elevates long-chain acyl-CoA (LC-CoA) levels in the hypothalamus.

67. The method of claim 66, wherein the composition reduces the activity of a CPT1.

68. A method of treating metabolic syndrome in a mammalian subject comprising intranasally administering to the mammalian subject an effective amount of a compound that elevates long-chain acyl-CoA (LC-CoA) levels in the hypothalamus.

69. The method of claim 68, wherein the compound reduces the activity of a CPT1.

70. A method of improving hepatic autoregulation in a mammalian subject comprising intranasally administering to the mammalian subject an effective amount of a compound that elevates long-chain acyl-CoA (LC-CoA) levels in the hypothalamus.

71. The method of claim 70, wherein the compound reduces the activity of a CPT1.

72. A method of reducing glucose production in a mammalian subject comprising intranasally administering to the mammalian subject an effective amount of a compound that elevates long-chain acyl-CoA (LC-CoA) levels in the hypothalamus.

73. The method of claim 72, wherein the compound reduces the activity of a CPT1.

74. A method of reducing food intake in a mammalian subject comprising intranasally administering to the mammalian subject an effective amount of a compound that elevates long-chain acyl-CoA (LC-CoA) levels in the hypothalamus.

75. The method of claim 74, wherein the compound reduces the activity of a CPT1.

76. A method of treating obesity in a mammalian subject comprising intranasally administering to the mammalian subject an effective amount of a compound that elevates long-chain acyl-CoA (LC-CoA) levels in the hypothalamus.

77. The method of claim 76, wherein the compound reduces the activity of a CPT1.
